(19) United States
(12) Patent Application Publication
(10) Pub. No.: US 2025/0129053 A1
(43) Pub. Date: Apr. 24, 2025

(54) HERBICIDAL IMIDAZOLE COMPOUNDS

(71) Applicant: SYNGENTA CROP PROTECTION AG, Basel (CH)

(72) Inventors: Suzanna Jane DALE, Bracknell, Berkshire (GB); Philip Michael ELVES, Bracknell, Berkshire (GB); Charles William Frederick KINGSTON, Bracknell, Berkshire (GB); James Alan MORRIS, Bracknell, Berkshire (GB); Samuel Vaughan WATKIN, Bracknell, Berkshire (GB)

(73) Assignee: SYNGENTA CROP PROTECTION AG, Basel (CH)

(21) Appl. No.: 18/702,511

(22) PCT Filed: Oct. 13, 2022
(86) PCT No.: PCT/EP2022/078500

§ 371 Date: Apr. 18, 2024

(30) Foreign Application Priority Data
Oct. 18, 2021 (GB) 2114863.0

(51) Int. Cl. C07D 403/06; A01N 25/32; A01N 43/54; A01P 13/00; C07D 401/14; C07D 403/14

(52) U.S. Cl. CPC C07D 403/06; A01N 25/32; A01N 43/54; A01P 13/00; C07D 401/14; C07D 403/14

(57) ABSTRACT

The present invention relates to compounds of Formula (I) or an agronomically acceptable salt of said compounds wherein Q, R¹ and R² are as defined herein. The invention further relates to herbicidal compositions which comprise a compound of Formula (I) and to the use of compounds of Formula (I) for controlling weeds, in particular in crops of useful plants.

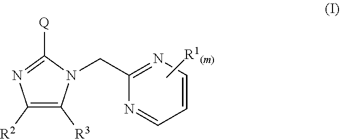

The present invention relates to novel herbicidal compounds, to processes for their preparation, to herbicidal compositions which comprise the novel compounds, and to their use for controlling weeds, in particular in crops of useful plants, or for inhibiting plant growth.

Thus, according to the present invention there is provided a compound of Formula (I):

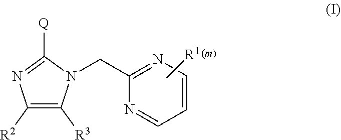

or an agronomically acceptable salt thereof,
wherein
Q is phenyl or a C-linked 6-membered heteroaryl wherein said phenyl or 6-membered heteroaryl is optionally substituted by one or more (e.g one or two) R⁴;
R¹ is independently selected from the group consisting of halogen, —CN, NO₂, C₁-C₄alkyl, C₁-C₄haloalkyl, C₃-C₆cycloalkyl, C₂-C₄alkenyl, C₂-C₄alkynyl, —S(O)ₚC₁-C₄alkyl, C₁-C₄alkoxy-, —C(O)C₁-C₄alkyl, —C(O)OC₁-C₄alkyl, C₁-C₄haloalkoxy and C₁-C₄alkoxyC₁-C₃alkyl-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —S(O)ₚC₁-C₄alkyl and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁵R⁶;
R⁴ is selected from the group consisting of halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁵R⁶;
R⁵ is hydrogen or C₁-C₄alkyl;
R⁶ is hydrogen or C₁-C₄alkyl;
m=1 or 2; and
p=0, 1 or 2.

C₁-C₄alkyl- and C₁-C₆alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

C₂-C₄alkenyl- includes, for example, —CH═CH₂ (vinyl) and —CH₂—CH═CH₂ (allyl).

C₂-C₄alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

Halogen (or halo) includes, for example, fluorine, chlorine, bromine or iodine. The same correspondingly applies to halogen in the context of other definitions, such as haloalkyl.

C₁-C₄haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl and heptafluoro-n-propyl. C₁-C₂haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₆alkoxy includes methoxy and ethoxy.

C₁-C₄haloalkoxy- includes, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₁-C₄alkoxyC₁-C₃alkyl- includes, for example, methoxymethyl-.

C₁-C₄alkoxyC₁-C₃alkoxy- includes, for example, methoxyethoxy-.

C₁-C₄alkoxyC₁-C₃alkoxyC₁-C₃alkyl- includes, for example, meth-oxyethoxymethyl-.

C₃-C₆cycloalkyl includes cyclopropyl, cyclopentyl and cyclohexyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention m is 1. In another embodiment of the present invention m is 2.

In one embodiment of the present invention, there is provided a compound of Formula (I) according to claim 1, wherein m is 1 and which is of Formula (Ia)

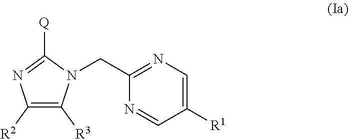

wherein Q, R¹, R² and R³ are as defined above.

In one embodiment of the present invention, R³ is hydrogen.

In a preferred embodiment of the present invention, R¹ is halogen (e.g chloro).

In another preferred embodiment of the present invention, R² is C₁-C₄haloalkyl (preferably —CF₃ or —CF₂H).

In another embodiment of the present invention, Q is selected from the group consisting of:

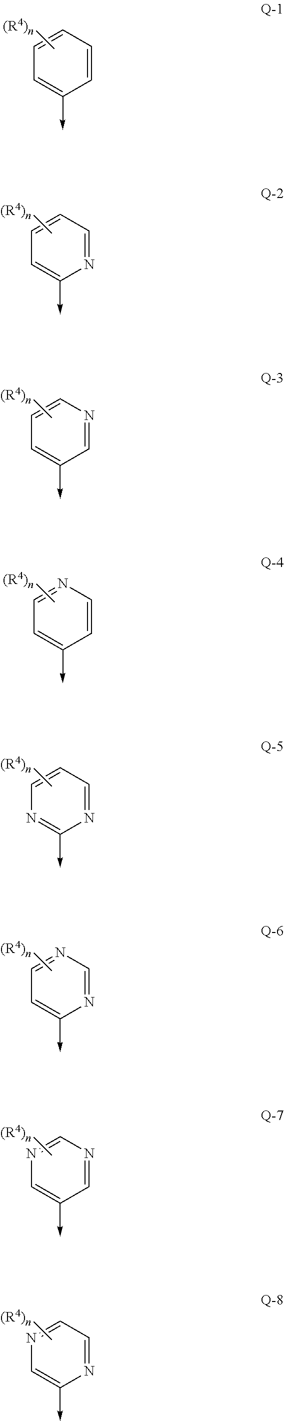

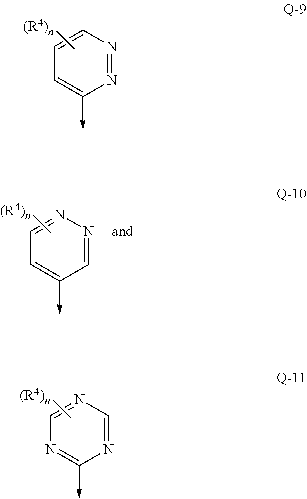

wherein n is 0, 1 or 2.

In a preferred embodiment of the present invention, Q is selected from the group consisting of Q-1, Q-3 and Q-7. Thus, in a more preferred embodiment of the present invention the compound of Formula (I) is selected from the group consisting of (Iaa), (Iab) and (Iac):

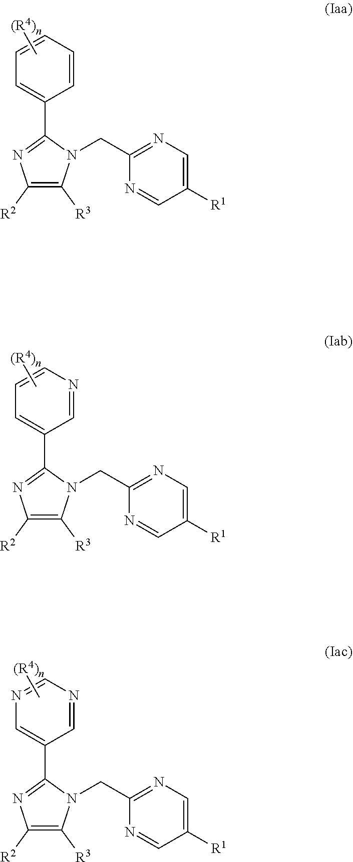

In another preferred embodiment, n is 1. In this embodiment, R⁴ is preferably selected from the group consisting of cyano, methyl, halogen and —CF₃. In a particularly preferred embodiment, Q is 4-Cl-phenyl-.

In another embodiment of the present invention, Q is Q-3 and n is 2. Thus, in a more preferred embodiment of the present invention the compound of Formula (I) is a compound of Formula (Iaba):

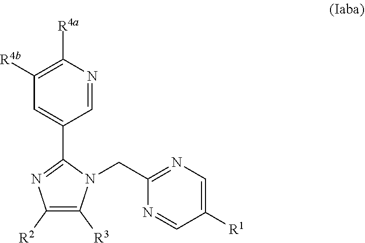

wherein R^{4a} is halogen, preferably fluoro or chloro and R^{4b}, preferably fluoro or chloro; and wherein R¹, R² and R³ are as defined in Formula (I). In a more preferred embodiment, there is provided a compound of Formula (Iaba) wherein R¹ is chloro, R² is —CF₃ or —CF₂H and R³ is hydrogen. Compounds of Formula (Iaba) are particularly preferred in the context of the present invention as they typically exhibit improved crop selectivity, particularly in maize.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]-imidazolidine-2-one, 5-ethoxy-4-hydroxy-1-methyl-3-[4-(trifluoromethyl)-2-pyridyl]-imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethyl-sulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonyl-methyl)-N-(5-methyl-1,3,4-oxadiazol-2-yl)-5-(trifluoromethyl)-[1,2,4]triazolo[4,3-a]-pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate, 6-chloro-4-(2,7-dimethyl-1-naphthyl)-5-hydroxy-2-methyl-pyridazin-3-one, tetrahydro-furan-2-ylmethyl(2R)-2-[(4-amino-3,5-dichloro-6-fluoro-2-pyridyl)oxy]-propanoate, (2R)-2-[(4-amino-3,5-dichloro-6-fluoro-2-pyridyl)oxy]propanoic acid, tetrahydrofuran-2-ylmethyl2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl6-amino-5-chloro-2-(4-chloro-2-fluoro-3-methoxyphenyl)-pyrimidine-4-carboxylate, 6-amino-5-chloro-2-(4-chloro-2-fluoro-3-methoxy-phenyl)-pyrimidine-4-carboxylic acid, 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl]N,N-diethylcarbamate.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds.

Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley soybean and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention can also be used in conjunction with crops that are tolerant to SDPS-inhibiting herbicides, such as those taught in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

### Processes for Preparation of Compounds of Formula (I)

Processes for preparation of compounds, e.g. a compound of formula (I) (which optionally can be an agrochemically acceptable salt thereof), are now described, and form further aspects of the present invention.

As shown in scheme 1 a compound of Formula (I) can be prepared by nucleophilic substitution by heating an aryl imidazole of Formula 2 in a suitable solvent, such as sulfolane or N,N-dimethylformamide in the presence of a base such as potassium or caesium carbonate with a compound of Formula 3 (where LG is halogen). The reaction is typically conducted at temperature ranging from 50 to 110° C. Conditions for the formation of imidazole compounds of Formula 2 are well documented in the literature (see for example Journal of Medicinal Chemistry, 2000, 43, 2165 and Synthetic Communications, 2020, 50, 700).

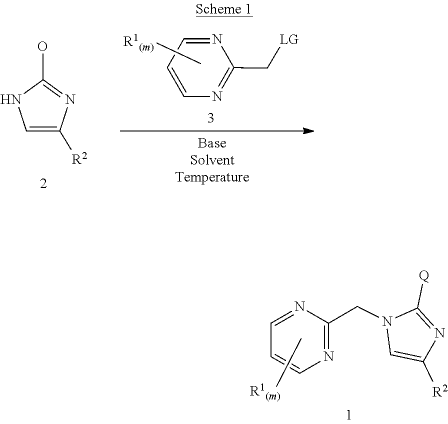

Alternatively, as shown in scheme 2, chemistry based on “C—H activation” can be used to prepare compounds of Formula (I). A compound of Formula 4 can be first be converted to a compound of Formula 6 by nucleophilic substitution by heating in a suitable solvent, such as acetonitrile or N,N-dimethylformamide in the presence of a base such as potassium or caesium carbonate with a compound of Formula 5 (where LG is halogen). The resulting alkylated imidazole of Formula 6 can then be arylated using C—H activation methods as outlined in Synlett, 2020, 31, 1015.

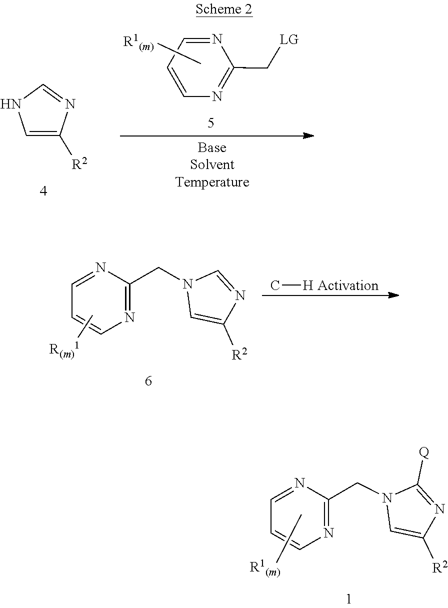

Compounds of Formula (I) can be further halogenated, with a suitable halogenating agent such as N-chlorosuccinimide, N-bromosuccinimide or N-iodosuccinimide in a suitable solvent such as acetonitrile. The halogens can then be further functionalised by methods reported in the literature (Bioorg Med Chem Lett. 2020; 30; 1269282; WO2020/132269, 2020)

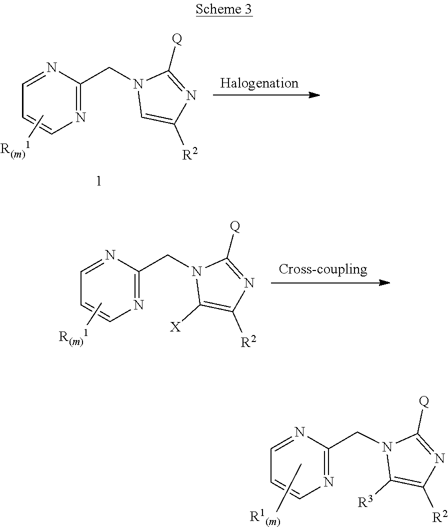

Alternatively, compounds of formula I, wherein Q, R², R³ and R¹₍ₘ₎ are as defined in formula I above can be prepared following scheme 4.

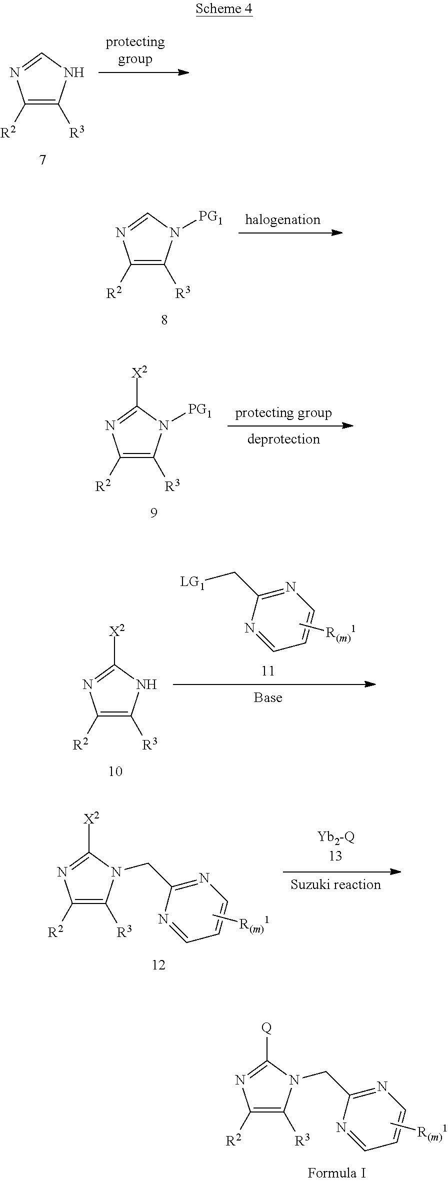

In scheme 4 compounds of formula I can be prepared by Suzuki cross-coupling reaction between compounds of formula 12, wherein X² is a leaving group like, for example, chlorine, bromine or iodine, with compounds of formula Yb₂-Q 13 wherein Q is as defined in formula I above and Yb₂ can be a boron-derived functional group, such as for example B(OH)₂ or B(OR_{b2})₂ wherein R_{b2} can be a C₁-C₄alkyl group or the two groups OR_{b2} can form together with the boron atom a five membered ring, as for example a pinacol boronic ester. The reaction may be catalyzed by a palladium based catalyst, for example tetrakis(triphenyl-phosphine)palladium(0), (1,1′bis(diphenylphosphino)ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, 2-methyl tetrahydrofuran, acetonitrile, N,N-dimethyl-formamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art.

Compounds of formula 12, can be prepared by alkylation reaction of compounds of formula 10, with compounds of formula 11, wherein LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art.

Compounds of formula 10 can be prepared by protecting group deprotection reaction from compounds of formula 9, wherein PG₁ is a N-protecting group for example acetyl, trimethylsilylethoxymethyl (SEM), tert-butyloxycarbonyl amongst others amino protecting groups. Such reactions are well known to those skilled in the art and can be carried out for example using base catalyzed or acid catalyzed such as HCl. Compounds of formula 9, can be prepared from compounds of formula 8 using halogenation reaction. Such reactions can be carried out in a two-step procedure which involved metalation using strong base such as butyl lithium, tert-butyl lithium, lithium tetramethylpiperidide, lithium diisopropylamide amongst other bases and quenching with suitably desired halogenating reagent such as molecular iodine, bromine or chlorine. Alternatively, halogenation reactions can be carried out in one step under radical conditions using halogenating reagent such as N-bromosuccinimide in the presence of a radical initiator such as azobisisobutyronitrile. Compounds of formula 8 can be prepared from compounds of formula 7 by protection group installation. Such reactions can be carried out in the presence of base such as sodium hydride, potassium carbonate, sodium carbonate, and in the presence of suitable protecting group reagents such as 2-(trimethylsilyl)ethoxymethyl chloride, acetyl chloride, di-tert-butyl dicarbonate and in the presence of solvent such as tetrahydrofuran, methanol, water, acetonitrile, dimethylformamide.

A compound of formula I-1 is a compound of Formula I, wherein R² is —CF₂H, R³ is —H and Q and R¹(m) are as defined in formula I. Compounds of formula I-1 can be prepared following scheme 5. In scheme 5 compounds of formula I-1 can be prepared by Suzuki cross-coupling reaction between compounds of formula 22, wherein X³ is a leaving group like, for example, chlorine, bromine or iodine, with compounds of formula Yb₃-Q 23 wherein Q is as defined in formula I above and Yb₃ can be a boron-derived functional group, such as for example B(OH)₂ or B(OR_{b2})₂ wherein R_{b2} can be a C₁-C₄alkyl group or the two groups OR_{b2} can form together with the boron atom a five membered ring, as for example a pinacol boronic ester following procedure as described in scheme 4 for the conversion of compounds of formula 12 to compounds of formula I.

Compounds of formula 22 can be prepared from compounds of formula 21 via fluorination reactions using fluorinating reagents such as diethylaminosulfur trifluoride or bis(2-methoxyethyl)aminosulfur trifluoride amongst others. Compounds of formula 21 can be prepared from compounds of formula 20 via oxidation reaction using oxidizing reagents such as MnO₂, SO₃·pyridine, pyridinium dichromate or pyridinium chlorochromate amongst other alcohol oxidizing reagents.

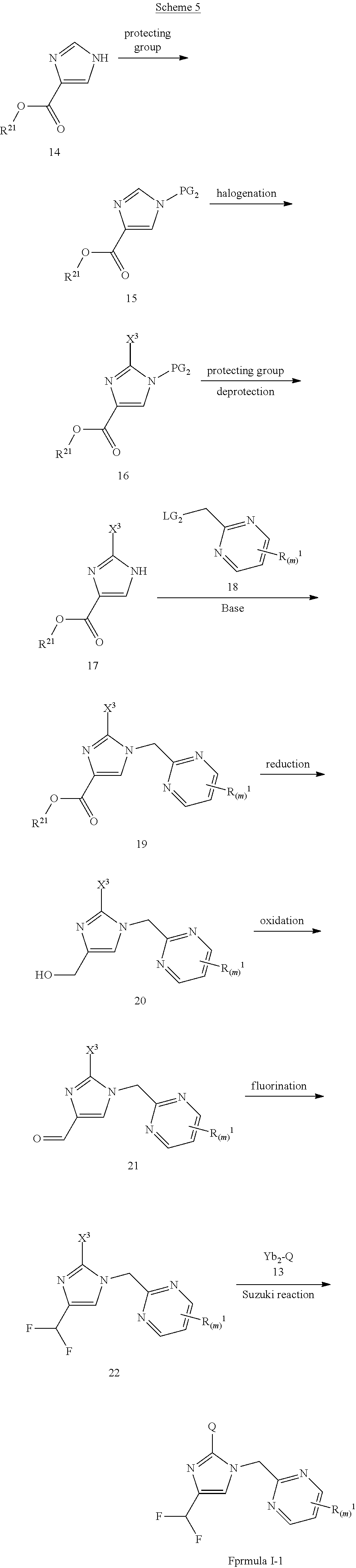

Alternatively compounds of formula 21 can be prepared following scheme 6.

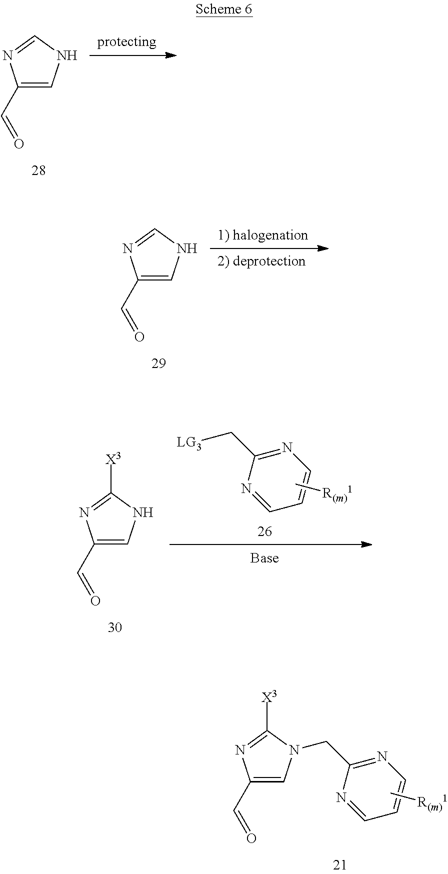

In scheme 6 compounds of formula 21 can be prepared by reacting compounds of formula 30 with compounds of formula 26 following procedure analogous to as described in scheme 4 for the conversion of compounds of formula 10 to compounds of formula 12. Compounds of formula 30 can be prepared from compounds of formula 29 via halogenation followed by N-deprotection reaction. Halogenation reactions can be carried out in one step under radical conditions using halogenating reagent such as N-bromosuccinimide in the presence of a radical initiator such as azobisisobutyronitrile. Deprotection reactions are well known to those skilled in the art and can be carried out for example using base catalysed or acid catalysed such as HCl. Compounds of formula 29 can be prepared from compound of formula 28 by protection group installation. Such reactions can be carried out in the presence of base such as sodium hydride, potassium carbonate, sodium carbonate, and in the presence of suitable protecting group reagents such as 2-(trimethylsilyl)ethoxymethyl chloride, acetyl chloride, di-tert-butyl dicarbonate and in the presence of solvent such as tetrahydrofuran, methanol, water, acetonitrile, dimethylformamide.

Compounds of formula 20 can be prepared from compounds of formula 19, wherein R²¹ is C₁-C₆alkyl via reduction reactions using reducing agents such as lithium aluminium hydride or diisobutylaluminium hydride. Compounds of formula 19 can be prepared by reacting compounds of formula 17 with compounds of formula 18, wherein LG₂ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art. Compounds of formula 17 can be prepared from compounds of formula 14 in three steps (scheme 5) following procedure analogous to as described in scheme 4 for the conversion of compounds of formula 7 to compounds of formula 10.

Alternatively compounds of formula I-1 can be prepared following scheme 7.

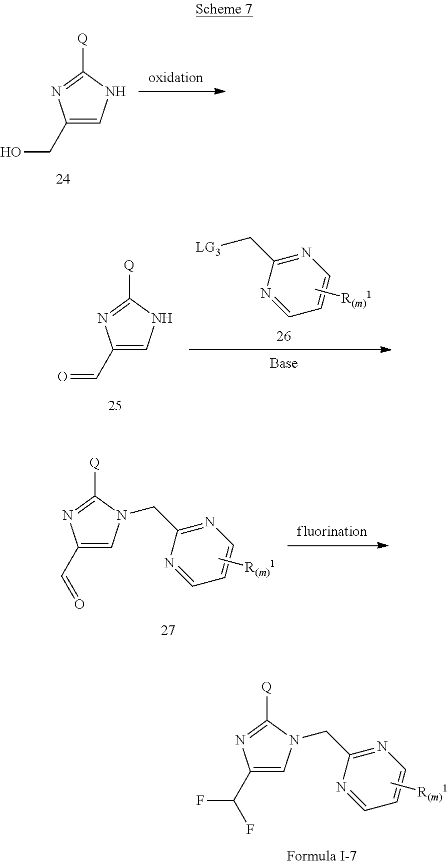

In scheme 7 compounds of formula I-1 can be prepared from compounds of formula 27 via fluorination reaction using fluorinating reagents such as diethylaminosulfur trifluoride or bis(2-methoxyethyl)aminosulfur trifluoride amongst others. Compounds of formula 27 can be prepared by reacting compounds of formula 25 with compounds of formula 26, wherein LG₃ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art. Compounds of formula 25 can be prepared from compounds of formula 24 via oxidation reaction using oxidizing agents such as MnO₂, SO₃·pyridine, pyridinium dichromate or pyridinium chlorochromate amongst other alcohol oxidizing reagents. Compounds of formula 24 can be prepared following procedure reported in literature for example in J. Med. Chem. 1995, 38, 2251-2255.

Alternatively compounds of formula 27 can be prepared following scheme 8. In scheme 8, compound of formula 27 can be prepared by reacting compounds of formula 25 with compounds of formula 26, following procedure analogous to as described in scheme 4 for the conversion of compounds of formula 10 to compounds of formula 12.

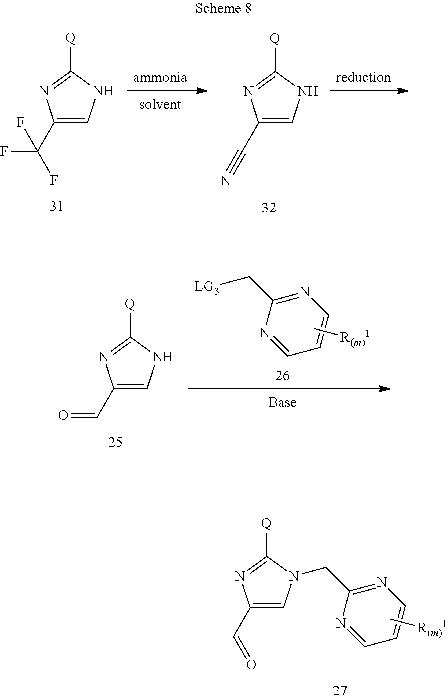

Compound of formula 25 can be prepared by reacting compound of formula 32 with a suitable reducing agent such as diisobutyl aluminium hydride. Compound of formula 32 can be prepared by reacting compound of formula 31 with ammonium hydroxide or similar other ammonia surrogates to transform the trifluoromethyl group to a cyano group. Such reactions are well documented in the literature (see for example Matthews, D. P.; Whitten, J. P.; McCarthy, J. R. J. Org. Chem. 1986, 51, 3228). Synthesis of imidazole compounds of Formula 31 are well documented in the literature (see for example Journal of Medicinal Chemistry, 2000, 43, 2165 and Synthetic Communications, 2020, 50, 700).

The following non-limiting examples provide specific synthesis methods for representative compounds of the present invention, as referred to in Table 1 below.

### EXAMPLE 1: 5-CHLORO-2-[[4-(TRIFLUOROMETHYL)-2-[4-(TRIFLUOROMETHYL)PHENYL]IMIDAZOL-1-YL]METHYL]PYRIMIDINE (COMPOUND 1.001)

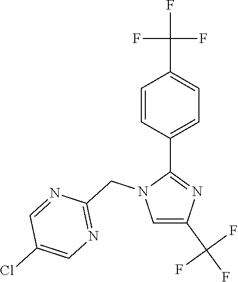 [SMILES: FC(F)(F)c1ccc(-c2nc(C(F)(F)F)cn2Cc2ncc(Cl)cn2)cc1]

### Step 1: Synthesis of 5-chloro-2-[[4-(trifluoromethyl)imidazol-1-yl]methyl]pyrimidine

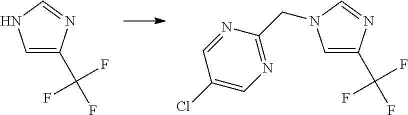 [SMILES: FC(F)(F)c1c[nH]cn1 ; FC(F)(F)c1cn(Cc2ncc(Cl)cn2)cn1]

A mixture of 4-(trifluoromethyl)-1H-imidazole (204 mg, 1.469 mmol), potassium carbonate (603 mg, 4.363 mmol) and 5-chloro-2-(chloromethyl)pyrimidine hydrochloride (365 mg, 1.738 mmol) in a mixture of acetonitrile (4.8 mL) and water (0.04 mL) was stirred at 80° C. for 22 hours. The reaction mixture was cooled, diluted with water, extracted with ethyl acetate and concentrated. The residues were subjected to flash column chromatography to give 5-chloro-2-[[4-(trifluoromethyl)imidazol-1-yl]methyl]pyrimidine (275 mg, 68%) as a yellow solid.

1H NMR (400 MHz, DMSO-d6) δ=8.96 (s, 2H), 7.94 (s, 1H), 7.88 (s, 1H), 5.58 (s, 2H)

### Step 2: Synthesis of 5-chloro-2-[[4-(trifluoromethyl)-2-[4-(trifluoromethyl)phenyl]imidazol-1-yl]methyl]pyrimidine (Compound 1.001)

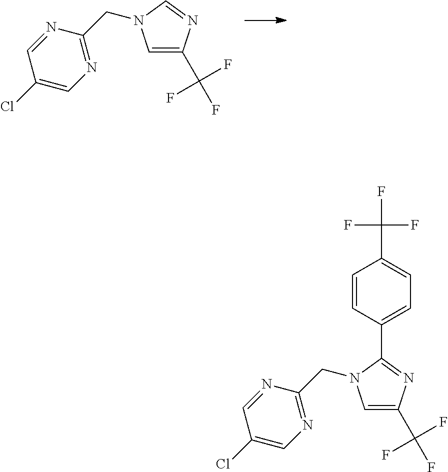 [SMILES: FC(F)(F)c1cn(Cc2ncc(Cl)cn2)cn1 ; FC(F)(F)c1ccc(-c2nc(C(F)(F)F)cn2Cc2ncc(Cl)cn2)cc1]

A mixture of 5-chloro-2-[[4-(trifluoromethyl)imidazol-1-yl]methyl]pyrimidine (48 mg, 0.174 mmol), 1-iodo-4-(trifluoromethyl)benzene (33 μL, 0.217 mmol), copper(I) iodide (69 mg, 0.362 mmol), palladium(II) acetate (3 mg, 0.013 mmol), triphenylphosphine (5 mg, 0.019 mmol) and DBU (56 μL, 0.361 mmol) in 1,4-dioxane (1.20 mL) was irradiated under microwave radiation to 140° C. for 2 hours. The reaction mixture was diluted with water, extracted with ethyl acetate and concentrated. The residues were subjected to flash column chromatography to give 5-chloro-2-[[4-(trifluoromethyl)-2-[4-(trifluoromethyl)phenyl]imidazol-1-yl]methyl]pyrimidine (33 mg, 44%) as an orange solid.

### EXAMPLE 2: 5-CHLORO-2-[[2-(4-FLUOROPHENYL)-4-(TRIFLUOROMETHYL)IMIDAZOL-1-YL]METHYL]PYRIMIDINE (COMPOUND 1.008)

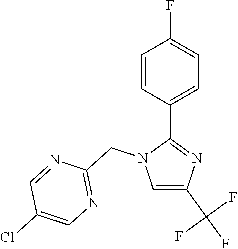 [SMILES: Fc1ccc(-c2nc(C(F)(F)F)cn2Cc2ncc(Cl)cn2)cc1]

### Step 1: Synthesis of 2-(4-fluorophenyl)-5-trifluoromethyl-1H-imidazole

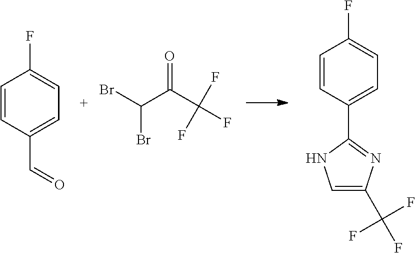 [SMILES: O=Cc1ccc(F)cc1 ; O=C(C(Br)Br)C(F)(F)F ; Fc1ccc(-c2nc(C(F)(F)F)c[nH]2)cc1]

A solution of sodium acetate (866 mg, 10.5 mmol) and 1,1-dibromo-3,3,3-trifluoroacetone (695 μL, 4.84 mmol) in water (4.0 mL) was stirred at 100° C. before being cooled to room temperature. The mixture was treated with aqueous ammonia (4.0 mL, 35 mmol, 35 mass %) and a solution of 4-fluorobenzaldehyde (430 μL, 4.01 mmol) in methanol (4.0 mL). The mixture was stirred at room temperature for 3 hours, diluted with water, extracted with ethyl acetate and concentrated. The residues were subjected to flash column chromatography to give 2-(4-fluorophenyl)-5-trifluoromethyl-1H-imidazole (488 mg, 50%) as a white solid.

1H NMR (400 MHz, chloroform) δ=9.65 (br s, 1H), 7.87-7.76 (m, 2H), 7.44 (s, 1H), 7.17-7.09 (m, 2H)

### Step 2: Synthesis of 5-chloro-2-[[2-(4-fluorophenyl)-4-(trifluoromethyl)imidazol-1-yl]methyl]pyrimidine (Compound 1.008)

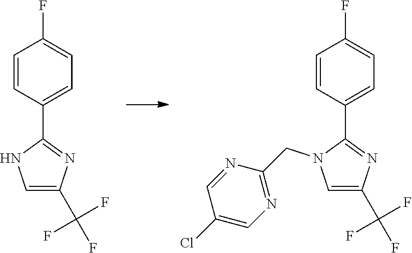 [SMILES: Fc1ccc(-c2nc(C(F)(F)F)c[nH]2)cc1 ; Fc1ccc(-c2nc(C(F)(F)F)cn2Cc2ncc(Cl)cn2)cc1]

A mixture of 2-(4-fluorophenyl)-4-(trifluoromethyl)-1H-imidazole (61 mg, 0.252 mmol), 5-chloro-2-(chloromethyl)pyrimidine hydrochloride (62 mg, 0.295 mmol) and potassium carbonate (111 mg, 0.803 mmol) in sulfolane (1.0 mL) was stirred at 100° C. for 3 hours. The reaction mixture was cooled, diluted with water, extracted with methyl tert-butyl ether and concentrated. The residues were subjected to reverse-phase column chromatography to give 5-chloro-2-[[2-(4-fluorophenyl)-4-(trifluoromethyl)imidazol-1-yl]methyl]pyrimidine (73 mg, 77%) as a beige solid

1H NMR (500 MHz, chloroform) δ=8.72 (s, 2H), 7.71-7.65 (m, 2H), 7.47 (s, 1H), 7.17-7.11 (m, 2H), 5.35 (s, 2H)

### EXAMPLE 3: 5-CHLORO-2-[[4,5-DICHLORO-2-[4-(TRIFLUOROMETHYL)PHENYL]IMIDAZOL-1-YL]METHYL]PYRIMIDINE (COMPOUND 1.013)

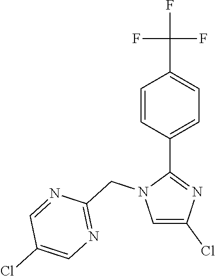 [SMILES: FC(F)(F)c1ccc(-c2nc(Cl)cn2Cc2ncc(Cl)cn2)cc1]

### Step 1: Synthesis of 5-chloro-2-[[4,5-dichloro-2-[4-(trifluoromethyl) phenyl]imidazol-1-yl]methyl]pyrimidine

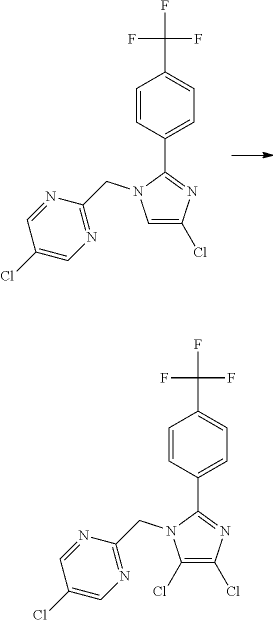 [SMILES: FC(F)(F)c1ccc(-c2nc(Cl)cn2Cc2ncc(Cl)cn2)cc1 ; FC(F)(F)c1ccc(-c2nc(Cl)c(Cl)n2Cc2ncc(Cl)cn2)cc1]

A solution of N-chlorosuccinimide (41 mg, 0.301 mmol) and 5-chloro-2-[[4-chloro-2-[4-(trifluoromethyl)phenyl]imidazol-1-yl]methyl]pyrimidine (44 mg, 0.112 mmol) in acetonitrile (0.5 mL) was stirred at 80° C. for 2 hours. The reaction mixture was cooled, diluted with water, extracted with ethyl acetate and concentrated. The residues were subjected to flash column chromatography to give 5-chloro-2-[[4,5-dichloro-2-[4-(trifluoromethyl)phenyl]imidazol-1-yl]methyl]pyrimidine (18 mg, 37%) as a yellow solid.

1H NMR (400 MHz, chloroform) δ=8.71 (s, 2H), 7.75-7.70 (m, 2H), 7.69-7.64 (m, 2H), 5.40 (s, 2H)

### EXAMPLE 4: PREPARATION OF 5-[1-[(5-CHLOROPYRIMIDIN-2-YL)METHYL]-4-(TRIFLUOROMETHYL)IMIDAZOL-2-YL]-3-METHYL-PYRIDINE-2-CARBONITRILE (1.016)

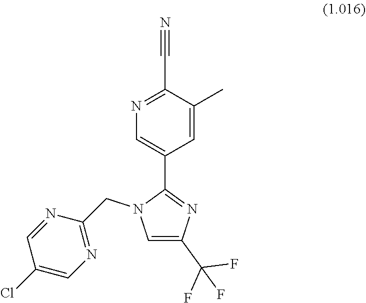 [SMILES: Cc1cc(-c2nc(C(F)(F)F)cn2Cc2ncc(Cl)cn2)cnc1C#N]

### Step 1: Preparation of trimethyl-[2-[[4-(trifluoromethyl)imidazol-1-yl]methoxy]ethyl]silane (I1)

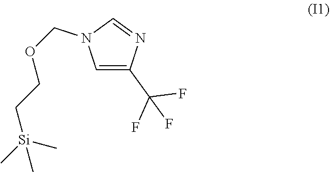 [SMILES: C[Si](C)(C)CCOCn1cnc(C(F)(F)F)c1]

To a suspension of 4-(trifluoromethyl)-1H-imidazole (3.07 g, 22.6 mmol) and potassium carbonate (9.14 g, 66.1 mmol) in acetonitrile (35.4 mL) was added 2-(chloromethoxy)ethyl-trimethyl-silane (5.8 mL, 33 mmol) and the resulting reaction mixture was stirred at room temperature overnight. The reaction mixture was charged with additional 2-(chloromethoxy)ethyl-trimethyl-silane (1.9 mL, 11 mmol) and stirred at room temperature for a further 4 hours. On completion, the mixture was diluted with brine and extracted with ethyl acetate. The combined organics were concentrated and subjected to column chromatography on silica gel using 0-60% ethyl acetate in cyclohexane to give trimethyl-[2-[[4-(trifluoromethyl)imidazol-1-yl]methoxy]ethyl]silane as a colourless oil I1 (2.74 g, 46% yield).

¹H NMR (400 MHz, CDCl₃) δ=7.65 (s, 1H), 7.42-7.36 (m, 1H), 5.31 (s, 2H), 3.63-3.34 (m, 2H), 1.02-0.83 (m, 2H), 0.01 (s, 9H)

### Step 2: Preparation of 2-[[2-iodo-4-(trifluoromethyl)imidazol-1-yl]methoxy]ethyl-trimethyl-silane (I2)

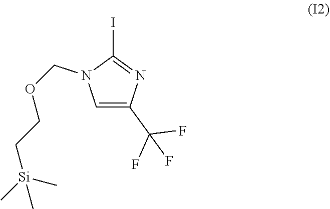 [SMILES: C[Si](C)(C)CCOCn1cc(C(F)(F)F)nc1I]

To a solution of trimethyl-[2-[[4-(trifluoromethyl)imidazol-1-yl]methoxy]ethyl]silane I-1 (5.31 g, 19.9 mmol) in tetrahydrofuran (100 mL) at −78° C. was added n-butyllithium (2.5 M in hexanes, 10.0 mL, 25 mmol). The reaction mixture was stirred for 30 minutes, treated with a solution of iodine (5.82 g, 22.9 mmol) in tetrahydrofuran (26 mL) and was allowed to stir for an hour. On completion the mixture was allowed to warm to room temperature before being quenched with aqueous sodium thiosulphate and extracted with ethyl acetate. The combined organics were washed with brine, concentrated and subjected to column chromatography on silica gel using 0-50% ethyl acetate in cyclohexane to give 2-[[2-iodo-4-(trifluoromethyl)imidazol-1-yl]methoxy]ethyl-trimethyl-silane I2 as a pale yellow solid (6.90 g, 88% yield). ¹H NMR (400 MHz, CDCl₃) δ=7.47 (q, 1H), 5.25 (s, 2H), 3.59-3.51 (m, 2H), 1.00-0.88 (m, 2H), 0.00 (s, 9H)

### Step 3: Preparation of 2-iodo-4-(trifluoromethyl)-1H-imidazole Hydrochloride (I3)

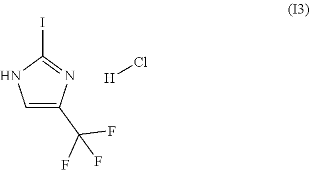

To a solution of 2-[[2-iodo-4-(trifluoromethyl)imidazol-1-yl]methoxy]ethyl-trimethyl-silane I-2 (7.2 g, 18 mmol) in ethanol (70 mL) was added hydrochloric acid (12 M in deionised water, 7.35 mL, 88 mmol). The mixture was stirred at 70° C. for 29 hours, cooled and concentrated to give 2-iodo-4-(trifluoromethyl)-1H-imidazole hydrochloride I-3 as a yellow solid (5.50 g, 95% yield). ¹H NMR (400 MHz, CD₃OD) δ=7.80 (q, 1H)

### Step 4: Preparation of 5-chloro-2-[[2-iodo-4-(trifluoromethyl)imidazol-1-yl]methyl]pyrimidine (I4)

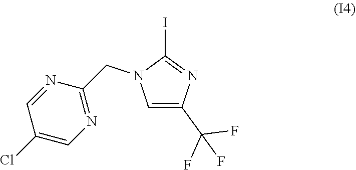 [SMILES: FC(F)(F)c1cn(Cc2ncc(Cl)cn2)c(I)n1]

To a mixture of 2-iodo-4-(trifluoromethyl)-1H-imidazole hydrochloride I-3 (348 mg, 1.166 mmol) and potassium iodide (37 mg, 0.223 mmol) was added acetonitrile (5.25 mL), potassium carbonate (536 mg, 3.878 mmol), water (0.18 mL) and 5-chloro-2-(chloromethyl)pyrimidine hydrochloride (279 mg, 1.329 mmol). The resulting mixture was stirred at 80° C. for one hour. The mixture was cooled, concentrated and subjected to column chromatography on silica gel using 0-100% ethyl acetate in cyclohexane to give 5-chloro-2-[[2-iodo-4-(trifluoromethyl)imidazol-1-yl]methyl]pyrimidine as a pale yellow solid I-4 (381 mg, 84% yield). ¹H NMR (400 MHz, CDCl₃) δ=8.69 (s, 2H), 7.51 (d, 1H), 5.34 (s, 2H).

### Step 5: Preparation of 5-[1-[(5-chloropyrimidin-2-yl)methyl]-4-(trifluoromethyl)imidazol-2-yl]-3-methyl-pyridine-2-carbonitrile (1.016)

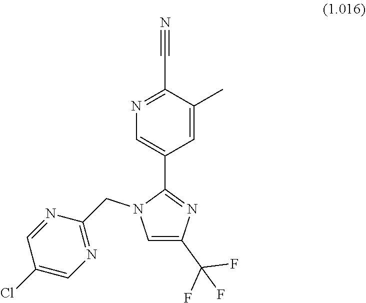 [SMILES: Cc1cc(-c2nc(C(F)(F)F)cn2Cc2ncc(Cl)cn2)cnc1C#N]

To a mixture of [1,1′-Bis(diphenylphosphino)ferrocene]dichloropalladium(II), complex with dichloromethane (37 mg, 0.0453 mmol), 4,4,5,5-tetramethyl-2-(4,4,5,5-tetramethyl-1,3,2-dioxaborolan-2-yl)-1,3,2-dioxaborolane (286 mg, 1.13 mmol), potassium acetate (221 mg, 2.25 mmol) and 5-bromo-3-methyl-pyridine-2-carbonitrile (180 mg, 0.914 mmol) was added 1,4-dioxane (2.4 mL) and the resulting reaction mixture was heated under microwave irradiation to 100° C. for an hour. To this mixture was added 5-chloro-2-[[2-iodo-4-(trifluoromethyl)imidazol-1-yl]methyl]pyrimidine I4 (246 mg, 0.633 mmol) as a solution in 1,4-dioxane (1 mL) and potassium phosphate tribasic (1.0 M in water, 1.60 mL, 1.60 mmol) and the reaction mixture was heated under microwave irradiation to 80° C. for a 8 hours. The mixture was diluted with brine and extracted with ethyl acetate. The combined organics were concentrated and subjected to column chromatography on silica gel using 0-50% ethyl acetate in cyclohexane. The residues were then subjected to reverse-phase column chromatography on C-18 silica gel using 30-70% acetonitrile in water, both with 0.1% formic acid, to give 5-[1-[(5-chloropyrimidin-2-yl)methyl]-4-(trifluoromethyl)imidazol-2-yl]-3-methyl-pyridine-2-carbonitrile 1.016 as a white solid (96 mg, 40% yield). ¹H NMR (400 MHz, CDCl3) δ=8.94 (d, 1H), 8.75 (s, 2H), 8.19 (d, 1H), 7.59 (d, 1H), 5.41 (s, 2H), 2.63 (s, 3H).

### EXAMPLE 5: PREPARATION OF 5-CHLORO-2-[[4-(DIFLUOROMETHYL)-2-(4-FLUOROPHENYL)IMIDAZOL-1-YL]METHYL]PYRIMIDINE (1.029)

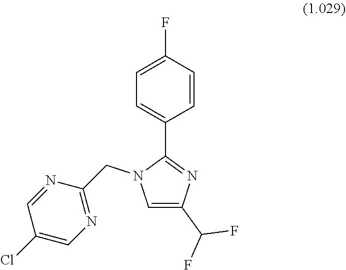 [SMILES: Fc1ccc(-c2nc(C(F)F)cn2Cc2ncc(Cl)cn2)cc1]

### Step 1: Preparation of [2-(4-fluorophenyl)-1H-imidazol-4-yl]methanol (I5)

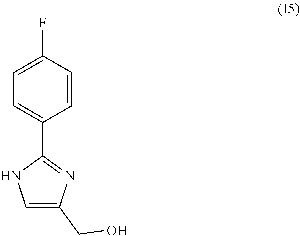 [SMILES: OCc1c[nH]c(-c2ccc(F)cc2)n1]

To a 500 mL round bottomed flask with stirrer bar was added 4-fluorobenzamidine hydrochloride (5.104 g, 28.35 mmol), 1,3-dihydroxyacetone dimer (5.4 g, 29 mmol), ammonium Hydroxide solution (140 mL, 35 mass %) and ammonium Chloride (6.23 g, 116 mmol). A condenser was fitted, and the stirred mixture was heated to 45° C. After 24 hours, the reaction was allowed to cool to room temperature. The reaction mixture was diluted with water (100 mL) and extracted with ethyl acetate (3×50 mL). The aqueous phase was acidified to pH 6 with 2M HCl (100 mL), and the aqueous phase was extracted with ethyl acetate (3×50 mL). The combined brown organic layers were concentrated in vacuo onto solid media for column chromatography (0-100% 3:1 ethyl acetate: ethanol in cyclohexane). Fractions were combined and concentrated in vacuo to provide [2-(4-fluorophenyl)-1H-imidazol-4-yl]methanol I5 (3.13 g, 90% purity, 52% Yield). ¹H NMR (400 MHz, DMSO-d₆) δ=12.62-12.21 (m, 1H), 8.07-7.88 (m, 2H), 7.36-7.19 (m, 2H), 7.11-6.80 (m, 1H), 5.23-4.76 (m, 1H), 4.42 (br s, 2H)

### Step 2: Preparation of 2-(4-fluorophenyl)-1H-imidazole-4-carbaldehyde I6

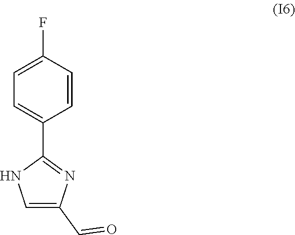 [SMILES: O=Cc1c[nH]c(-c2ccc(F)cc2)n1]

To a 100 mL round bottomed flask with stirrer bar was added [2-(4-fluorophenyl)-1H-imidazol-4-yl]methanol I5 (540 mg, 2.5287 mmol), manganese(IV) oxide (3.03 g, 34.5 mmol) and ethyl acetate (25 mL, 255 mmol). The mixture was stirred at room temperature. After 48 hours, the reaction mixture was filtered through Kieselguhr and washed with ethyl acetate (20 mL). The yellow filtrate was concentrated in vacuo. The solid was redissolved in ethyl acetate and concentrated in vacuo onto solid media for column chromatography (40-100% ethyl acetate in cyclohexane) Fractions were combined and concentrated in vacuo to yield a pale yellow solid (249 mg, 49%, 95% purity) 2-(4-fluorophenyl)-1H-imidazole-4-carbaldehyde I6 (249 mg, 49% Yield) ¹H NMR (400 MHz, DMSO-d₆) δ=13.46 (br s, 1H), 9.75 (s, 1H), 8.11 (m, 3H), 7.35 (m, 2H)

### Step 3: Preparation of 1-[(5-chloropyrimidin-2-yl)methyl]-2-(4-fluorophenyl)imidazole-4-carbaldehyde (I7)

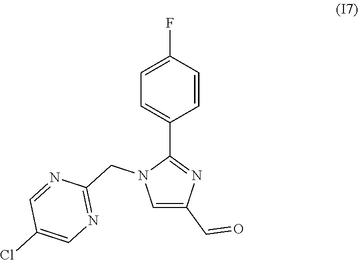 [SMILES: O=Cc1cn(Cc2ncc(Cl)cn2)c(-c2ccc(F)cc2)n1]

To a microwave vial with stirrer bar was added 2-(4-fluorophenyl)-1H-imidazole-4-carbaldehyde I6 (249 mg, 1.2439 mmol), 5-chloro-2-(chloromethyl)pyrimidine hydrochloride (341 mg, 1.6241 mmol), potassium carbonate (434 mg, 3.1401 mmol), potassium iodide (42 mg, 0.25 mmol), acetonitrile (3.8 mL) and water (0.38 mL). The mixture was heated to 90° C. for 2 hours in the microwave. The reaction mixture was cooled to room temperature, diluted with water (20 mL) and extracted with ethyl acetate (3×10 mL). The combined orange organic layers were concentrated in vacuo onto solid media for column chromatography (30-80% ethyl acetate in cyclohexane). Fractions were combined and concentrated in vacuo to give 1-[(5-chloropyrimidin-2-yl)methyl]-2-(4-fluorophenyl)imidazole-4-carbaldehyde I7 (273 mg, 66% yield). ¹H NMR (400 MHz, chloroform) δ=9.94 (s, 1H), 8.71 (s, 2H), 7.84 (s, 1H), 7.67 (m, 2H), 7.15 (m, 2H), 5.40 (s, 2H).

### Step 4: Preparation of 5-chloro-2-[[4-(difluoromethyl)-2-(4-fluorophenyl)imidazol-1-yl]methyl]pyrimidine (1.029)

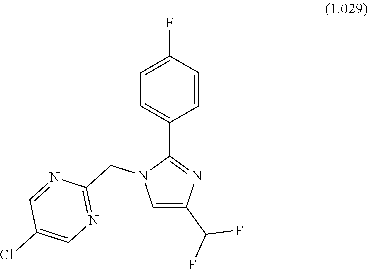 [SMILES: Fc1ccc(-c2nc(C(F)F)cn2Cc2ncc(Cl)cn2)cc1]

To a 50 mL three necked round bottomed flask with stirrer bar was added 1-[(5-chloropyrimidin-2-yl)methyl]-2-(4-fluorophenyl)imidazole-4-carbaldehyde I7 (273 mg, 0.8189 mmol). The flask was evacuated and backfilled three times with nitrogen, before the addition of dichloromethane (15 mL, 234 mmol) and diethylaminosulfur trifluoride (0.9 mL, 7 mmol). The mixture was stirred at room temperature for 67 hours. The reaction was quenched with saturated sodium bicarbonate solution (30 mL) and extracted with ethyl acetate (3×10 mL). The combined orange organic layers were concentrated in vacuo onto solid media for column chromatography (0-40% ethyl acetate in cyclohexane) Fractions were combined and concentrated in vacuo to yield an orange gel 5-chloro-2-[[4-(difluoromethyl)-2-(4-fluorophenyl)imidazol-1-yl]methyl]pyrimidine (152.4 mg, 52% Yield). ¹H NMR (400 MHz, chloroform) δ=8.71 (s, 2H), 7.67 (m, 2H), 7.37 (t, 1H), 7.13 (t, 2H), 6.72 (t, 1H), 5.35 (s, 2H).

### EXAMPLE 6: PREPARATION OF 5-CHLORO-2-[[2-(6-CHLORO-5-FLUORO-3-PYRIDYL)-4-(TRIFLUOROMETHYL)IMIDAZOL-1-YL]METHYL]PYRIMIDINE (1.037)

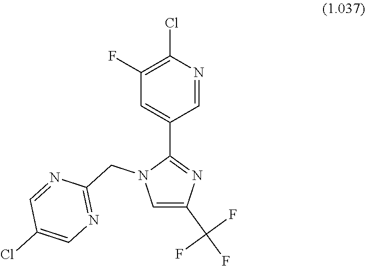 [SMILES: Fc1cc(-c2nc(C(F)(F)F)cn2Cc2ncc(Cl)cn2)cnc1Cl]

### Step 1: Preparation of 2-chloro-3-fluoro-5-[4-(trifluoromethyl)-1H-imidazol-2-yl]pyridine I8

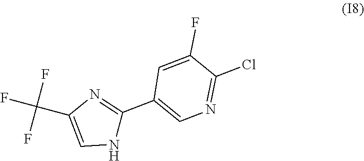 [SMILES: Fc1cc(-c2nc(C(F)(F)F)c[nH]2)cnc1Cl]

To a round bottomed flask with stirrer bar was added sodium acetate hydrate (1.207 g, 14.42 mmol), water (5.8 mL, 99 mass %) and 1,1,-dibromo-3,3,3-trifluoroacetone (0.995 mL, 6.94 mmol). A condenser was fitted, and the reaction mixture was heated to 90° C. for 30 minutes and then cooled to give a solution of 3,3,3-trifluoro-2-oxo-propanal. To a separate round bottomed flask with stirrer bar was added 6-chloro-5-fluoro-pyridine-3-carbaldehyde (971 mg, 5.7816 mmol), methanol (15 mL) and aqueous ammonia (5.8 mL, 51 mmol, 35 mass %) at 0° C. The reaction mixture was stirred at 0° C. for 30 minutes to which was added dropwise a solution of 3,3,3-trifluoro-2-oxo-propanal (prepared above) and reaction mixture was stirred for additional 2 h and then allowed to warm to room temperature and left stirring for 20 h. Reaction mixture was diluted with water (30 mL) and extracted with ethyl acetate (3×20 mL). Organic layers were collected and concentrated in vacuo and purified via column chromatography (10% ethyl acetate in cyclohexane). Fractions were combined and concentrated in vacuo to give 2-chloro-3-fluoro-5-[4-(trifluoromethyl)-1H-imidazol-2-yl]pyridine I8 (794 mg, 49%). ¹H NMR (400 MHz, DMSO-d₆) δ=13.60 (br s, 1H), 8.86 (dd, 1H), 8.37 (dd, 1H), 8.13-8.09 (m, 1H).

### Step 2: Preparation of 5-chloro-2-[[2-(6-chloro-5-fluoro-3-pyridyl)-4-(trifluoromethyl)imidazol-1-yl]methyl]pyrimidine (1.037)

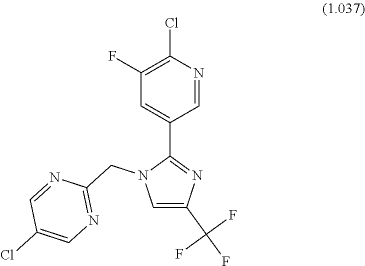 [SMILES: Fc1cc(-c2nc(C(F)(F)F)cn2Cc2ncc(Cl)cn2)cnc1Cl]

To a microwave vial with stirrer bar was added 2-chloro-3-fluoro-5-[4-(trifluoromethyl)-1H-imidazol-2-yl]pyridine I8 (485 mg, 1.7348 mmol), 5-chloro-2-(chloromethyl)pyrimidine hydrochloride (450 mg, 2.1432 mmol), potassium carbonate (598 mg, 4.3267 mmol), acetonitrile (10 mL), potassium iodide (56 mg, 0.3373433 mmol) and water (1 mL). The mixture was heated to 70° C. in the microwave for 2 hours. The reaction mixture was diluted with water (30 mL) and extracted with ethyl acetate (3×10 mL). The combined organic layers were concentrated in vacuo onto solid media for column chromatography (0-40% ethyl acetate in cyclohexane). Fractions were combined and concentrated in vacuo to give 5-chloro-2-[[2-(6-chloro-5-fluoro-3-pyridyl)-4-(trifluoromethyl)imidazol-1-yl]methyl]pyrimidine 1.037 (634 mg, 89%). ¹H NMR (400 MHz, chloroform) δ=8.74 (s, 2H), 8.63 (d, 1H), 8.04 (dd, 1H), 7.55 (q, 1H), 5.39 (s, 2H).

### EXAMPLE 7: PREPARATION OF 5-CHLORO-2-[[2-(6-CHLORO-5-FLUORO-3-PYRIDYL)-4-(DIFLUOROMETHYL)IMIDAZOL-1-YL]METHYL]PYRIMIDINE (1.026)

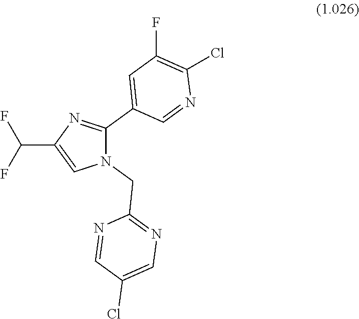 [SMILES: Fc1cc(-c2nc(C(F)F)cn2Cc2ncc(Cl)cn2)cnc1Cl]

### Step 1—Preparation of methyl 1-(2-trimethylsilylethoxymethyl)imidazole-4-carboxylate I9

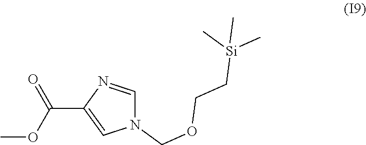

To a solution of methyl 1H-imidazole-4-carboxylate (5.00 g, 39.6 mmol) in MeCN (75 mL) was added K₂CO₃ (13.7 g, 99.1 mmol). 2-(chloromethoxy)ethyl-trimethyl-silane (10 mL, 57 mmol) was then added dropwise via dropping funnel maintaining an internal temperature of the reaction mixture below 30° C. After complete addition, the reaction mixture stirred rapidly at room temperature for 4 h. The reaction was quenched with sat. NaCl (75 mL) and EtOAc (50 mL) was added. The phases were separated, and the aqueous phase was extracted with EtOAc (3×50 mL). The organics were combined, washed with sat. NaCl and concentrated onto granulated celite. The crude product was purified by flash chromatography on silica gel using a gradient of 0-100% EtOAc in cyclohexane as eluent to give methyl 1-(2-trimethylsilylethoxymethyl)imidazole-4-carboxylate I9 (2.56 g, 25%) as a pale yellow oil. ¹H NMR (400 MHz, CDCl₃) δ=7.74 (d, 1H), 7.63 (d, 1H), 5.32 (s, 2H), 3.92 (s, 3H), 3.57-3.43 (m, 2H), 1.04-0.75 (m, 2H), 0.00 (s, 9H)

### Step 2—Preparation of methyl 2-bromo-1-(2-trimethylsilylethoxymethyl)imidazole-4-carboxylate I10

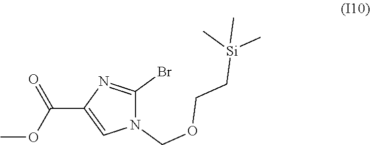

To a solution of methyl 1-(2-trimethylsilylethoxymethyl)imidazole-4-carboxylate I9 (2.02 g, 7.88 mmol) and N-bromosuccinimide (1.84 g, 10.1 mmol) in MeCN (20 mL) was added AIBN (0.1 g, 0.60 mmol) and the reaction was heated to 60° C. under N₂ for 3 h. The reaction was quenched with 2M sodium thiosulphate (10 mL), and EtOAc (20 mL) and Brine (20 mL) were added. The phases were separated, and the aqueous phase was extracted with EtOAc (3×50 mL). The organics were combined, washed with sat. NaCl and concentrated onto granulated celite. The crude product was purified by flash chromatography on silica gel using a gradient of 0-100% EtOAc in cyclohexane as eluent to give methyl 2-bromo-1-(2-trimethylsilylethoxymethyl)imidazole-4-carboxylate I10 (1.57 g, 59%) as a colourless oil. ¹H NMR (400 MHz, chloroform) δ=7.78 (s, 1H), 5.31 (s, 2H), 3.91 (s, 3H), 3.56 (m, 2H), 0.94 (m, 2H), 0.01 (s, 9H).

### Step 3—Preparation of methyl 2-bromo-1H-imidazole-4-carboxylate; hydrochloride I11

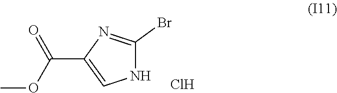

To a solution of methyl 2-bromo-1-(2-trimethylsilylethoxymethyl)imidazole-4-carboxylate I10 (1.57 g, 4.68 mmol) in ethanol (40 mL) was added 12M HCl (4.00 mL). The reaction was heated to 60° C. under N₂ for 6 h. The reaction mixture was concentrated under vacuum to give methyl 2-bromo-1H-imidazole-4-carboxylate; hydrochloride I11 (1.15 g, 96% Yield). ¹H NMR (400 MHz, MeOD) δ=8.02 (s, 1H), 3.91 (s, 3H)

### Step 4—Preparation of methyl 2-bromo-1-[(5-chloropyrimidin-2-yl)methyl]imidazole-4-carboxylate I12

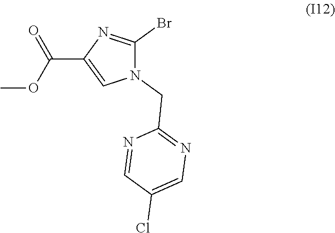 [SMILES: COC(=O)c1cn(Cc2ncc(Cl)cn2)c(Br)n1]

To a stirred suspension of methyl 2-bromo-1H-imidazole-4-carboxylate; hydrochloride I11 (1.15 g, 4.52 mmol) and KI (0.15 g, 0.90 mmol) in MeCN (20 mL) was added K₂CO₃ (2.19 g, 15.8 mmol) and H₂O (1.50 mL). 5-chloro-2-(chloromethyl)pyrimidine hydrochloride (1.14 g, 5.43 mmol) was added and the reaction was heated to 80° C. for 2 h. The reaction was quenched with sat. NaCl (10 mL) and EtOAc (10 mL) was added. The phases were separated, and the aqueous phase was extracted with EtOAc (3×50 mL). The organics were combined, washed with sat. NaCl and concentrated onto granulated celite. The crude product was purified by flash chromatography on silica gel using a gradient of 0-100% EtOAc in cyclohexane as eluent to give methyl 2-bromo-1-[(5-chloropyrimidin-2-yl)methyl]imidazole-4-carboxylate I12 (1.36 g, 86%). ¹H NMR (400 MHz, CDCl₃) δ=8.68 (s, 2H), 7.80 (s, 1H), 5.38 (s, 2H), 3.90 (s, 3H)

### Step 5—Preparation of [2-bromo-1-[(5-chloropyrimidin-2-yl)methyl]imidazol-4-yl]methanol I13

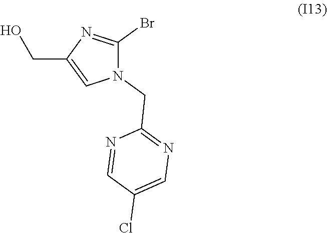 [SMILES: OCc1cn(Cc2ncc(Cl)cn2)c(Br)n1]

To a solution of methyl 2-bromo-1-[(5-chloropyrimidin-2-yl)methyl]imidazole-4-carboxylate I12 (1.36 g, 3.90 mmol) in 2-methyltetrahydrofuran (20 mL) at 0° C. under N₂ was added DIBAL-H 1.0 M in hexane (11.70 mL, 11.70 mmol) dropwise. The reaction was stirred at 0° C. for 1 h then warmed to room temperature. The reaction was stirred at room temperature for 16 h. The reaction was diluted with methyl tert-butyl ether (10 mL), cooled to 0° C. and quenched with 467 μL of water, 467 μL of 15% NaOH in water and 1.17 mL of water was then added. The reaction was then warmed to room temperature and stirred at room temperature for 30 minutes. Celite and MgSO₄ were added, and the reaction mixture filtered through a sintered funnel under suction. The filtrate was concentrated under vacuum to give [2-bromo-1-[(5-chloropyrimidin-2-yl)methyl]imidazol-4-yl]methanol I13 (1.16 g, 88%). ¹H NMR (400 MHz, CDCl₃) δ=8.68 (s, 2H), 7.08 (s, 1H), 5.32 (m, 2H), 4.59 (s, 2H), 2.15 (s, 1H).

### Step 6—Preparation of 2-bromo-1-[(5-chloropyrimidin-2-yl)methyl]imidazole-4-carbaldehyde I14

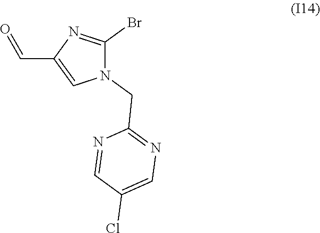 [SMILES: O=Cc1cn(Cc2ncc(Cl)cn2)c(Br)n1]

To a solution of [2-bromo-1-[(5-chloropyrimidin-2-yl)methyl]imidazol-4-yl]methanol I13 (1.16 g, 3.44 mmol) in 2-methyltetrahydrofuran (20 mL) at room temperature was added MnO₂ (4.49 g, 51.6 mmol) and the reaction stirred at room temperature for 24 h. MnO₂ (1.50 g, 17.3 mmol) was added and the reaction mixture stirred at room temperature for 48 h. The reaction was diluted with EtOAc (100 mL) and filtered through a pad of celite. The filtrate was concentrated under vacuum to give 2-bromo-1-[(5-chloropyrimidin-2-yl)methyl]imidazole-4-carbaldehyde I14 (0.77 g, 75%). ¹H NMR (400 MHz, CDCl₃) δ=9.82 (s, 1H), 8.69 (s, 2H), 7.80 (s, 1H), 5.40 (s, 2H).

### Step 7—Preparation of 2-[[2-bromo-4-(difluoromethyl)imidazol-1-yl]methyl]-5-chloro-pyrimidine I15

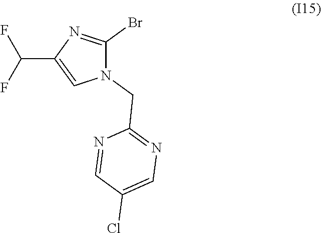 [SMILES: FC(F)c1cn(Cc2ncc(Cl)cn2)c(Br)n1]

To a solution of 2-bromo-1-[(5-chloropyrimidin-2-yl)methyl]imidazole-4-carbaldehyde I14 (0.77 g, 2.56 mmol) in dichloromethane (50 mL) cooled to 0° C. was added diethylaminosulfurtrifluoride (2.72 mL, 20.6 mmol) dropwise. The reaction was stirred rapidly at 0° C. under N₂ for 3 h. The reaction was quenched with sat. NaHCO₃ (50 mL) and dichloromethane (25 mL) was added. The phases were separated, and the organics concentrated onto granulated celite. The crude product was purified by flash chromatography on silica gel using a gradient of 0-100% EtOAc in cyclohexane as eluent to give 2-[[2-bromo-4-(difluoromethyl)imidazol-1-yl]methyl]-5-chloro-pyrimidine I15 (0.45 g, 54%) as a brown oil. ¹H NMR (400 MHz, CDCl₃) δ=8.69 (s, 2H), 7.37 (t, 1H), 6.64 (t, 1H), 5.36 (s, 2H).

### Step 8—Preparation of 5-chloro-2-[[2-(6-chloro-5-fluoro-3-pyridyl)-4-(difluoromethyl)imidazol-1-yl]methyl]pyrimidine 1.026

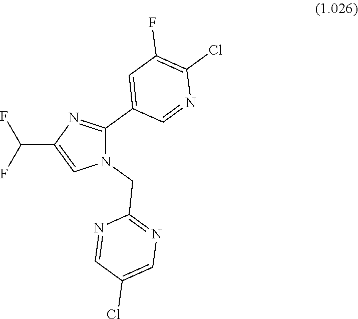 [SMILES: Fc1cc(-c2nc(C(F)F)cn2Cc2ncc(Cl)cn2)cnc1Cl]

To a microwave vial was added 2-[[2-bromo-4-(difluoromethyl)imidazol-1-yl]methyl]-5-chloro-pyrimidine I15 (0.040 g, 0.12 mmol), 2-chloro-3-fluoro-5-(4,4,5,5-tetramethyl-1,3,2-dioxaborolan-2-yl)pyridine (0.041 g, 0.16 mmol) and (1,1′-Bis(diphenylphosphino)ferrocene)palladium(II) dichloride (5 mg, 0.006 mmol). The vessel placed under an atmosphere of N₂ and sealed. 2-Methyltetrahydrofuran (0.50 mL) and Potassium Phosphate Tribasic (1.0 M in Water) (0.30 mL, 0.30 mmol) were added and the reaction mixture heated to 100° C. for 1 h under microwave irradiation. The reaction was cooled to room temperature and water (10 mL) and EtOAc (10 mL) were added. The phases were separated, and the aqueous phase extracted with EtOAc (3×5 mL). The organics were combined and concentrated onto celite. The crude product was purified by flash chromatography on silica gel using a gradient of 0-100% EtOAc/cyclohexane as eluent followed by purification by flash chromatography on C18 reverse-phase silica using a gradient of 30-100% MeCN in Water (with 0.1% formic acid modifier) as eluent to give 5-chloro-2-[[2-(6-chloro-5-fluoro-3-pyridyl)-4-(difluoromethyl)imidazol-1-yl]methyl]pyrimidine 1.026 (14 mg, 31%) as a colourless oil. ¹H NMR (400 MHz, MeOD) δ=8.81 (s, 2H), 8.51 (d, 1H), 8.09 (dd, 1H), 7.69 (t, 1H), 6.77 (t, 1H), 5.56 (s, 2H).

### EXAMPLE 8: SYNTHESIS OF 5-CHLORO-2-[[5-CHLORO-2-(6-CHLORO-3-PYRIDYL)-4-(DIFLUOROMETHYL)IMIDAZOL-1-YL]METHYL]PYRIMIDINE 1.076

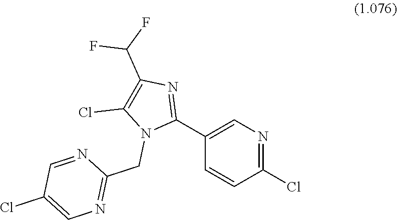 [SMILES: FC(F)c1nc(-c2ccc(Cl)nc2)n(Cc2ncc(Cl)cn2)c1Cl]

### Step 1: Synthesis of 2-chloro-5-[4-(trifluoromethyl)-1H-imidazol-2-yl]pyridine I16

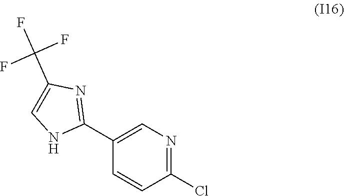 [SMILES: FC(F)(F)c1c[nH]c(-c2ccc(Cl)nc2)n1]

To a solution of sodium acetate (1.5 g, 18 mmol) in water (10 mL) was added 1,1-dibromo-3,3,3-trifluoroacetone 3,3-dibromo-1,1,1-trifluoro-propan-2-one (2.3 g, 8.5 mmol). The reaction mixture was heated to 100° C. for 30 minutes. The reaction was then cooled to room temperature. To this solution was added aqueous ammonia (5.0 ml) and then 6-chloropyridine-3-carbaldehyde (1.0 g, 7.1 mmol) in methanol (20 ml) slowly and stirred for 14 hours. The reaction mixture was quenched with water (100 mL), extracted in ethyl acetate (3×250 mL), dried over anhydrous sodium sulfate, filtered and concentrated under reduced pressure. The residue was purified by (200-400) silica using the ethyl acetate and cyclohexane (20:80) to afford 2-chloro-5-[4-(trifluoromethyl)-1H-imidazol-2-yl]pyridine 116 as a pale yellow solid (1.0 g, 57%). ¹H NMR (400 MHz, CHLOROFORM-d) δ ppm 8.96 (d, 1H) 8.32 (dd, 1H) 7.32-7.44 (m, 2H).

### Step 2: Synthesis of 2-(6-chloro-3-pyridyl)-1H-imidazole-4-carbonitrile I17

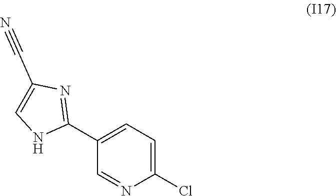 [SMILES: N#Cc1c[nH]c(-c2ccc(Cl)nc2)n1]

A mixture of 2-chloro-5-[4-(trifluoromethyl)-1H-imidazol-2-yl]pyridine 116 (0.5 g, 2.02 mmol), methanol (2.5 ml) and aqueous ammonia (4.8 ml, 30.291 mmol) was heated at 70° C. in a sealed tube for 10 hours. The reaction mixture was cooled to room temperature and evaporated under reduced pressure. The residue was purified by (200-400) silica using ethyl acetate and cyclohexane (40:60) to afford 2-(6-chloro-3-pyridyl)-1H-imidazole-4-carbonitrile I17 (0.4 g, 96%). ¹H NMR (400 MHz, METHANOL-d4) δ ppm 8.93 (dd, 1H), 8.32 (dd, 1H), 8.03 (s, 1H), 7.61 (dd, 1H).

### Step 3: Synthesis of 2-(6-chloro-3-pyridyl)-1H-imidazole-4-carbaldehyde I18

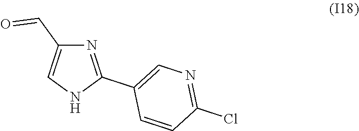 [SMILES: O=Cc1c[nH]c(-c2ccc(Cl)nc2)n1]

To a stirred solution of 2-(6-chloro-3-pyridyl)-1H-imidazole-4-carbonitrile I17 (0.51 g, 2.4924 mmol) in dry tetrahydrofuran (10.2 ml) at −78° C. was added diisobutyl aluminium hydride in toluene (12 mL, 12.46 mmol, 1 molar solution) dropwise. Then the reaction mass was stirred at same temperature for an hour and allowed to come to 0° C. and stirred for 1 hr. The reaction mass was quenched with 2N HCl and extracted with ethyl acetate. Then organic layer was washed well with water, brine, dried on sodium sulphate and concentrated to get crude material. The residue was purified by (200-400) silica using the ethyl acetate and cyclohexane (30:70) to afford 2-(6-chloro-3-pyridyl)-1H-imidazole-4-carbaldehyde I18 (160 mg, 30%). ¹H NMR (400 MHz, METHANOL-d4) δ ppm 9.83 (s, 1H), 8.98 (d, 1H), 8.38 (dd, 1H), 8.08 (br s, 1H), 7.63 (d, 1H).

### Step 4: Synthesis of 2-(6-chloro-3-pyridyl)-1-[(5-chloropyrimidin-2-yl)methyl]imidazole-4-carbaldehyde I19

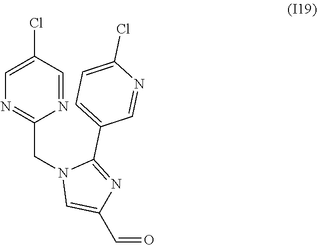 [SMILES: O=Cc1cn(Cc2ncc(Cl)cn2)c(-c2ccc(Cl)nc2)n1]

To a solution of 2-(6-chloro-3-pyridyl)-1H-imidazole-4-carbaldehyde I18 (0.16 g, 0.77 mmol) in acetonitrile (1.6 mL) was added potassium carbonate (0.26 g, 1.92 mmol), potassium iodide (0.025 g, 0.15 mmol) and 5-chloro-2-(chloromethyl)pyrimidine (0.15 g, 0.92 mmol) and heated to 70° C. for 16 h. The reaction mixture was diluted with water (100 mL) and extracted with ethyl acetate. The combined organic layer was washed with brine, dried over sodium sulphate and concentrated under reduced pressure to obtain the crude. The residue was purified by (200-400) silica using the ethyl acetate and cyclohexane (20:80) to afford 2-(6-chloro-3-pyridyl)-1-[(5-chloropyrimidin-2-yl)methyl]imidazole-4-carbaldehyde I19 (0.2 g, 78%). ¹H NMR (400 MHz, METHANOL-d4) δ ppm 9.72 (s, 1H), 8.69 (s, 1H), 8.66-8.69 (m, 1H), 8.55 (dd, 1H), 8.10 (s, 1H), 7.96-8.01 (m, 1H), 7.46 (dd, 1H), 5.50 (s, 2H).

### Step 5: Synthesis of 5-chloro-2-[[2-(6-chloro-3-pyridyl)-4-(difluoromethyl)imidazol-1-yl]methyl]pyrimidine 1.028

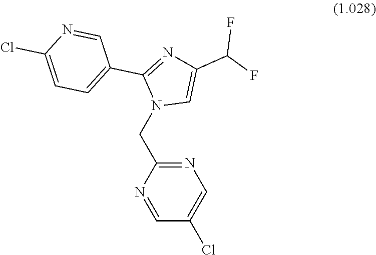 [SMILES: FC(F)c1cn(Cc2ncc(Cl)cn2)c(-c2ccc(Cl)nc2)n1]

To a solution of 2-(6-chloro-3-pyridyl)-1-[(5-chloropyrimidin-2-yl)methyl]imidazole-4-carbaldehyde I19 (0.200 g, 0.56 mmol) in dichloromethane (12 mL), was added diethylaminosulfur trifluoride (0.6 mL, 4.5 mmol) and stirred at room temperature for 16 h. The reaction mixture was quenched with sodium bicarbonate solution (50 mL) and extracted with ethyl acetate. The combined organic layers were dried over sodium sulphate and concentrated under reduced pressure to obtain the crude. The residue was purified by (200-400) silica using ethyl acetate and cyclohexane (20:80) to afford 5-chloro-2-[[2-(6-chloro-3-pyridyl)-4-(difluoromethyl)imidazol-1-yl]methyl]pyrimidine 1.028 (120 mg, 56%). ¹H NMR (400 MHz, CHLOROFORM-d) δ ppm 8.72 (s, 2H), 8.09 (dd, 1H), 7.43-7.47 (m, 2H), 7.27 (s, 1H), 6.73 (t, 1H), 5.37 (s, 2H).

### Step 6: Synthesis of 5-chloro-2-[[5-chloro-2-(6-chloro-3-pyridyl)-4-(difluoromethyl)imidazol-1-yl]methyl]pyrimidine 1.076

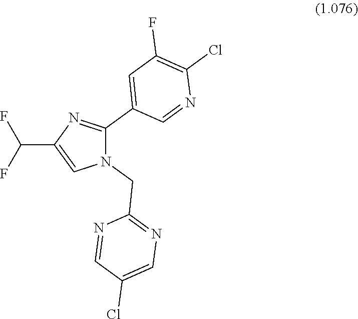 [SMILES: Fc1cc(-c2nc(C(F)F)cn2Cc2ncc(Cl)cn2)cnc1Cl]

To a solution of 5-chloro-2-[[2-(6-chloro-3-pyridyl)-4-(difluoromethyl)imidazol-1-yl]methyl]pyrimidine 1.028 (0.07 g, 0.19 mmol) in acetonitrile (1.4 mL) was added N-chlorosuccinimide (0.035 g, 0.25 mmol) and stirred at room temperature for 16 h. After the completion of reaction, the reaction mass was quenched with 10% sodium thiosulphate solution (100 mL) and extracted with ethyl acetate, dried over sodium sulphate and concentrated to get crude material. The residue was purified by (200-400) silica using the ethyl acetate and cyclohexane (20:80) to afford 5-chloro-2-[[5-chloro-2-(6-chloro-3-pyridyl)-4-(difluoromethyl)imidazol-1-yl]methyl]pyrimidine 1.076 as off-white solid (45 mg, 58%). ¹H NMR (400 MHz, METHANOL-d4) δ ppm 8.72 (s, 2H), 8.51 (dd, 1H), 7.94 (dd, 1H), 7.45 (dd, 1H), 6.72 (t, 1H), 5.43 (s, 2H).

### EXAMPLE 9: SYNTHESIS OF 5-CHLORO-2-[[4-(DIFLUOROMETHYL)-2-(2-METHYLPYRIMIDIN-5-YL)IMIDAZOL-1-YL]METHYL]PYRIMIDINE 1.077

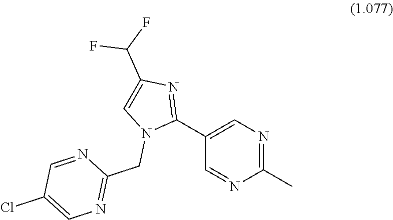 [SMILES: Cc1ncc(-c2nc(C(F)F)cn2Cc2ncc(Cl)cn2)cn1]

### Step 1: Synthesis of 1-(2-trimethylsilylethoxymethyl)imidazole-4-carbaldehyde and 3-(2-trimethylsilylethoxymethyl)imidazole-4-carbaldehyde (I20+I21)

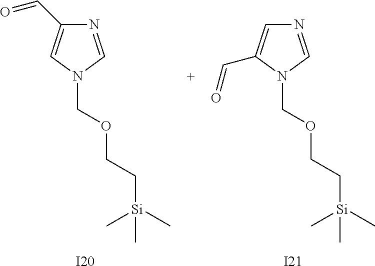

To a solution of 1H-imidazole-4-carbaldehyde (4.0 g, 42 mmol) in tetrahydrofuran (42 mL) was added sodium hydride (1.2 equiv., 50 mmol, 60 mass %) at 0° C. The mixture was stirred at 0° C. for 15 min. Then 2-(trimethylsilyl)ethoxymethyl chloride (8.5 g, 46 mmol) was added dropwise. After the addition, the mixture was stirred at 25° C. for 10 h. The reaction mass was cooled to <5° C., quenched with saturated ammonium chloride and extracted with ethyl acetate (200 mL×3). Then organic layer was washed with brine solution, dried over anhydrous sodium sulphate and concentrated to get crude material. The residue was purified by (200-400) silica using the ethyl acetate and cyclohexane (30:70) to afford 1-(2-trimethylsilylethoxymethyl)imidazole-4-carbaldehyde (7.65 g, 81% Yield) as a mixture of I20 and I21 with its isomer 3-(2-trimethylsilylethoxymethyl)imidazole-4-carbaldehyde in the ratio 2:1 respectively. Major isomer ¹H NMR (400 MHz, CDCl₃) δ ppm 9.90 (s, 1H), 7.73 (d, 1H), 7.69 (s, 1H), 5.33 (s, 2H), 3.47-3.54 (m, 2H), 0.85-0.97 (m, 2H), −0.05-0.01 (m, 9H). Minor isomer ¹H NMR (400 MHz, CDCl₃) δ ppm 9.79 (s, 1H), 7.87 (s, 1H), 7.82 (s, 1H), 5.69 (s, 1H), 3.59-3.65 (m, 2H), 0.85-0.97 (m, 2H), −0.05-0.01 (m, 9H).

### Step 2: Synthesis of 2-bromo-1-(2-trimethylsilylethoxymethyl)imidazole-4-carbaldehyde and 2-bromo-3-(2-trimethylsilylethoxymethyl)imidazole-4-carbaldehyde (I22+I23)

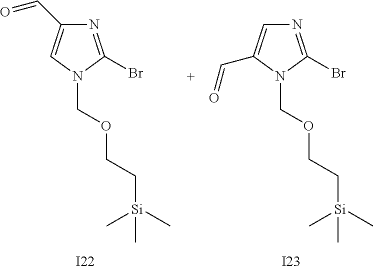

To a stirred solution of mixture of 1-(2-trimethylsilylethoxymethyl)imidazole-4-carbaldehyde I20 and I21 (2.5 g, 11 mmol) in carbon tetrachloride (50 mL) was added azobisisobutyronitrile (0.093 g, 0.55 mmol) followed by N-bromosuccinimide (2.2 g, 12 mmol) at room temperature. The reaction mass was heated at 85° C. for 16 h. The reaction mass was cooled to room temperature, quenched with 10% aqueous solution of sodium thiosulphate and layers were separated. The aqueous layer was extracted ethyl acetate (200 ml×3). The combined organic layer was washed with brine solution, dried over sodium sulphate and concentrated to give mixture of 2-bromo-1-(2-trimethylsilylethoxymethyl)imidazole-4-carbaldehyde I22 and 2-bromo-3-(2-trimethylsilylethoxymethyl)imidazole-4-carbaldehyde 123 (3.0 g, 9.8 mmol, 89%) as gummy mass as crude material. Major isomer ¹H NMR (400 MHz, DMSO-d6) δ ppm 9.66 (s, 1H), 8.38 (s, 1H), 5.38 (s, 2H), 3.47-3.61 (m, 2H), 0.82-0.90 (m, 2H), −0.10-0.04 (m, 9H) and Minor isomer ¹H NMR (400 MHz, DMSO-d6) δ ppm 9.62 (s, 1H), 8.36 (s, 1H), 5.41 (m, 2H), 3.47-3.61 (m, 2H), 0.82-0.90 (m, 2H), −0.10-0.04 (m, 9H)

### Step 3: Synthesis of 2-bromo-1H-imidazole-4-carbaldehyde I24

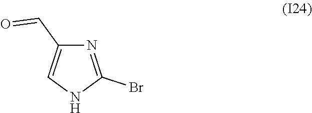

A mixture of 2-bromo-1-(2-trimethylsilylethoxymethyl)imidazole-4-carbaldehyde I22 and I23 (1.1 g, 3.6 mmol) and 2,2,2-trifluoroacetic acid (4.2 mL, 54 mmol) was stirred at room temperature for 16 h. The reaction mixture was poured into ice cold water, added solid sodium bicarbonate under stirring until the pH becomes neutral. The reaction mass was stirred for 15 min, then extracted with ethyl acetate (100 mL×5). The combined organic layer was washed with brine solution, dried over sodium sulphate, and concentrated to get crude material. The residue was purified by (200-400) silica using the ethyl acetate and cyclohexane (30:70) to afford 2-bromo-1H-imidazole-4-carbaldehyde I24 (0.545 g, 2.96 mmol, 82%) as gummy mass, which upon standing became solid. ¹H NMR (400 MHz, CD₃OD) δ ppm 9.64 (s, 1H), 7.78-7.95 (m, 1H).

### Step 4: Synthesis of 2-bromo-1-[(5-chloropyrimidin-2-yl)methyl]imidazole-4-carbaldehyde I25

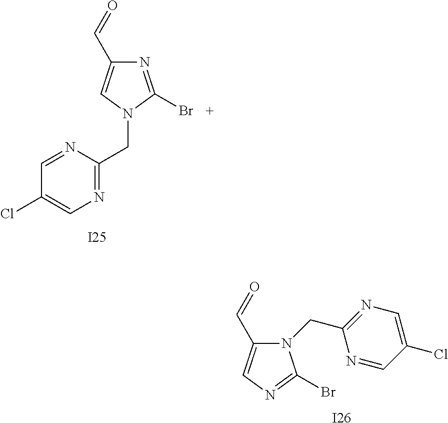 [SMILES: "I25" ; O=Cc1cn(Cc2ncc(Cl)cn2)c(Br)n1 ; "I26" ; O=Cc1cnc(Br)n1Cc1ncc(Cl)cn1]

To a solution of 2-bromo-1H-imidazole-4-carbaldehyde I24 (0.4 g, 2.28 mmol) in acetonitrile (9.14 mL) was added potassium carbonate (0.63 g, 4.57 mmol), potassium iodide (0.037 g, 0.23 mmol) and 5-chloro-2-(chloromethyl)pyrimidine (0.45 g, 2.51 mmol). The reaction mixture heated to 60° C. for 16 h. The reaction mixture was diluted with water (100 mL) and extracted with ethyl acetate (100 mL×3). The combined organic layer was washed with brine solution, dried over anhydrous sodium sulphate and concentrated under reduced pressure to obtain the crude material. The residue was purified by (200-400) silica using the ethyl acetate and cyclohexane (30:70) to afford a mixture of 2-bromo-1-[(5-chloropyrimidin-2-yl)methyl]imidazole-4-carbaldehyde I25 and 2-bromo-3-[(5-chloropyrimidin-2-yl)methyl]imidazole-4-carbaldehyde 126 (0.455 g, 1.49 mmol, 65%). Major isomer 125 ¹H NMR (400 MHz, CDCl₃) δ ppm 9.81 (s, 1H), 8.68 (s, 2H), 7.80 (s, 1H), 5.40 (s, 2H). Minor isomer I26 ¹H NMR (400 MHz, CDCl₃) δ ppm 9.80 (s, 1H), 8.60 (s, 2H), 7.75 (s, 1H), 5.39 (s, 2H).

### Step 5: Synthesis of 2-[[2-bromo-4-(difluoromethyl)imidazol-1-yl]methyl]-5-chloro-pyrimidine I27

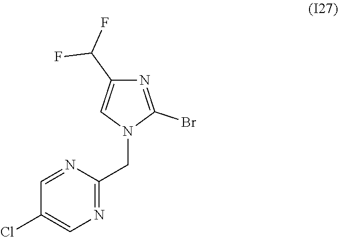 [SMILES: FC(F)c1cn(Cc2ncc(Cl)cn2)c(Br)n1]

To a solution of 2-bromo-1-[(5-chloropyrimidin-2-yl)methyl]imidazole-4-carbaldehyde I25 (0.45 g, 1.49 mmol) in dichloromethane (30 mL) at 0° C. was added diethylaminosulfur trifluoride (1.66 mL, 11.94 mmol) dropwise. The mixture was stirred at room temperature for 16 h. The reaction mixture was cooled to 0° C., slowly quenched with sodium bicarbonate solution (30 mL) and extracted with ethyl acetate (3×100 mL). The combined organic layers were dried over sodium sulphate and concentrated under reduced pressure to obtain the crude. The residue was purified by (200-400) silica using ethyl acetate and cyclohexane (25:75) to afford 2-[[2-bromo-4-(difluoromethyl)imidazol-1-yl]methyl]-5-chloro-pyrimidine I27 (0.234 g, 0.72 mmol, 48%) as light brown thick liquid. ¹H NMR (400 MHz, CDCl₃) δ ppm 8.68 (s, 2H), 7.36 (t, 1H), 6.62 (t, 1H), 5.35 (s, 2H).

### Step 6: Synthesis of 5-chloro-2-[[4-(difluoromethyl)-2-(2-methylpyrimidin-5-yl)imidazol-1-yl]methyl]pyrimidine 1.077

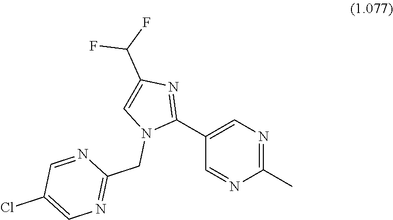 [SMILES: Cc1ncc(-c2nc(C(F)F)cn2Cc2ncc(Cl)cn2)cn1]

To a 10 mL microwave vial was added 2-[[2-bromo-4-(difluoromethyl)imidazol-1-yl]methyl]-5-chloro-pyrimidine I27 (0.05 g, 0.15 mmol) and (2-methylpyrimidin-5-yl)boronic acid (0.064 g, 0.46 mmol) in 1,4-dioxane (0.5 mL). To this mixture was added potassium phosphate tribasic (0.084 g, 0.386 mmol) and [1,1′-Bis(diphenylphosphino)ferrocene]dichloropalladium(II) (0.016 g, 0.0196 mmol) and heated at 100° C. for 1 h. After the completion of reaction, the reaction mass was diluted with 5 ml water, extracted with ethyl acetate, washed with brine solution, dried over sodium sulphate and concentrated to get crude material. The residue was purified by (200-400) silica using the ethyl acetate and cyclohexane (30:70) to afford 5-chloro-2-[[4-(difluoromethyl)-2-(2-methylpyrimidin-5-yl)imidazol-1-yl]methyl]pyrimidine 1.077 (0.03 g, 0.089 mmol, 58%) was obtained as gummy mass. ¹H NMR (400 MHz, CDCl₃) δ ppm 9.02 (s, 2H), 8.70 (s, 2H), 7.45 (s, 1H), 6.71 (t, 1H), 5.34 (s, 2H), 2.77-2.81 (s, 3H).

TABLE 1

| | | 1^{H} NMR (CDCl₃ unless stated |
|---|---|---|
| Compound | STRUCTURE | otherwise) |
| 1.001 | 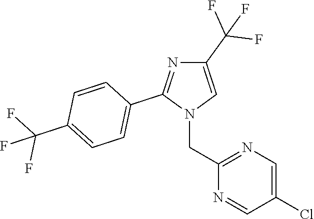 [SMILES: FC(F)(F)c1ccc(-c2nc(C(F)(F)F)cn2Cc2ncc(Cl)cn2)cc1] | 1H NMR (400 MHz, chloroform) δ = 8.73 (s, 2H), 7.86 (d, 2H), 7.72 (d, 2H), 7.54-7.51 (m, 1H), 5.39 (s, 2H) |
| 1.002 | 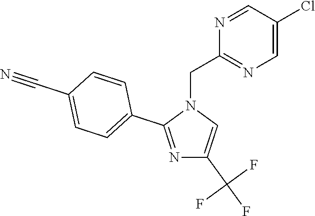 [SMILES: N#Cc1ccc(-c2nc(C(F)(F)F)cn2Cc2ncc(Cl)cn2)cc1] | 1H NMR (500 MHz, chloroform) δ = 8.73 (s, 2H), 7.89 (d, 2H), 7.75 (d, 2H), 7.54 (s, 1H), 5.39 (s, 2H) |
| 1.003 | 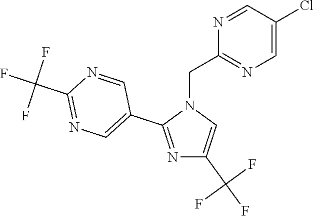 [SMILES: FC(F)(F)c1cn(Cc2ncc(Cl)cn2)c(-c2cnc(C(F)(F)F)nc2)n1] | 1H NMR (400 MHz, chloroform) δ = 9.41 (s, 2H), 8.75 (s, 2H), 7.64-7.62 (m, 1H), 5.41 (s, 2H) |
| 1.004 | 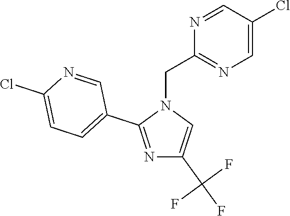 [SMILES: FC(F)(F)c1cn(Cc2ncc(Cl)cn2)c(-c2ccc(Cl)nc2)n1] | 1H NMR (500 MHz, chloroform) δ = 8.77 (d, 1H), 8.72 (s, 2H), 8.09 (dd, 1H), 7.54 (s, 1H), 7.45 (d, 1H), 5.36 (s, 2H) |
| 1.005 | 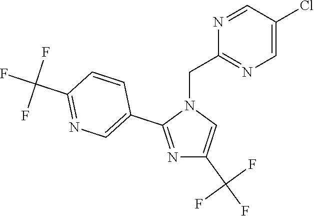 [SMILES: FC(F)(F)c1ccc(-c2nc(C(F)(F)F)cn2Cc2ncc(Cl)cn2)cn1] | 1H NMR (400 MHz, chloroform) δ = 9.13 (d, 1H), 8.74 (s, 2H), 8.35 (dd, 1H), 7.81 (d, 1H), 7.58 (d, 1H), 5.40 (s, 2H) |
| 1.006 | 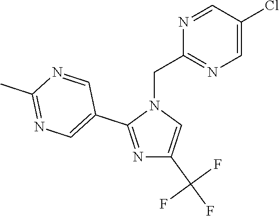 [SMILES: Cc1ncc(-c2nc(C(F)(F)F)cn2Cc2ncc(Cl)cn2)cn1] | 1H NMR (400 MHz, chloroform) δ = 9.04 (s, 2H), 8.72 (s, 2H), 7.56 (q, 1H), 5.35 (s, 2H), 2.81 (s, 3H) |
| 1.007 | 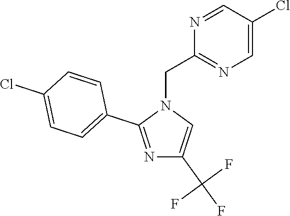 [SMILES: FC(F)(F)c1cn(Cc2ncc(Cl)cn2)c(-c2ccc(Cl)cc2)n1] | 1H NMR (500 MHz, chloroform) δ = 8.72 (s, 2H), 7.67-7.61 (m, 2H), 7.48 (s, 1H), 7.46-7.39 (m, 2H), 5.35 (s, 2H) |
| 1.008 | 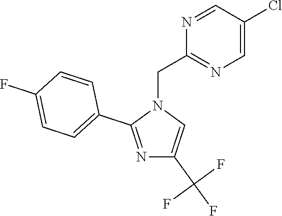 [SMILES: Fc1ccc(-c2nc(C(F)(F)F)cn2Cc2ncc(Cl)cn2)cc1] | 1H NMR (500 MHz, chloroform) δ = 8.72 (s, 2H), 7.71-7.65 (m, 2H), 7.47 (s, 1H), 7.17-7.11 (m, 2H), 5.35 (s, 2H) |
| 1.009 | 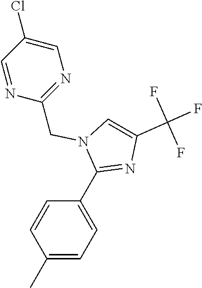 [SMILES: Cc1ccc(-c2nc(C(F)(F)F)cn2Cc2ncc(Cl)cn2)cc1] | 1H NMR (400 MHz, chloroform) δ = 8.71 (s, 2H), 7.53 (d, 2H), 7.46 (s, 1H), 7.24 (d, 2H), 5.37 (s, 2H), 2.39 (s, 3H) |
| 1.010 | 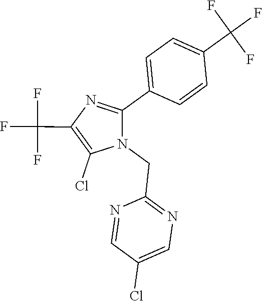 [SMILES: FC(F)(F)c1ccc(-c2nc(C(F)(F)F)c(Cl)n2Cc2ncc(Cl)cn2)cc1] | 1H NMR (400 MHz, chloroform) δ = 8.71 (s, 2H), 7.73 (d, 2H), 7.68 (d, 2H), 5.41 (s, 2H) |
| 1.011 | 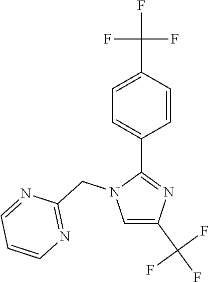 [SMILES: FC(F)(F)c1ccc(-c2nc(C(F)(F)F)cn2Cc2ncccn2)cc1] | 1H NMR (400 MHz, chloroform) δ = 8.78 (d, 2H), 7.89 (d, 2H), 7.71 (d, 2H), 7.57-7.54 (m, 1H), 7.33 (t, 1H), 5.40 (s, 2H) |
| 1.012 | 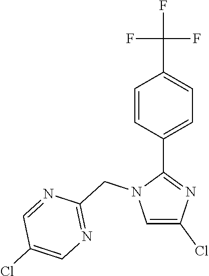 [SMILES: FC(F)(F)c1ccc(-c2nc(Cl)cn2Cc2ncc(Cl)cn2)cc1] | 1H NMR (400 MHz, chloroform) δ = 8.72 (s, 2H), 7.85 (d, 2H), 7.70 (d, 2H), 7.07 (s, 1H), 5.34 (s, 2H) |
| 1.013 | 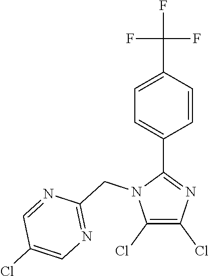 [SMILES: FC(F)(F)c1ccc(-c2nc(Cl)c(Cl)n2Cc2ncc(Cl)cn2)cc1] | 1H NMR (400 MHz, chloroform) δ = 8.71 (s, 2H), 7.75-7.70 (m, 2H), 7.69- 7.64 (m, 2H), 5.40 (s, 2H) |
| 1.014 | 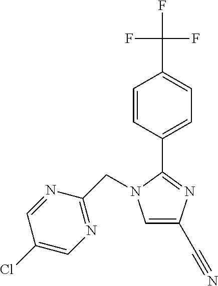 [SMILES: N#Cc1cn(Cc2ncc(Cl)cn2)c(-c2ccc(C(F)(F)F)cc2)n1] | 1H NMR (400 MHz, chloroform) δ = 8.73 (s, 2H), 7.82 (d, 2H), 7.77-7.70 (m, 3H), 5.40 (s, 2H) |
| 1.015 | 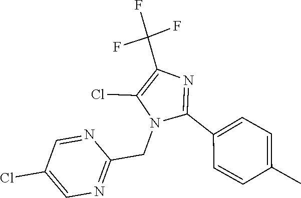 [SMILES: Cc1ccc(-c2nc(C(F)(F)F)c(Cl)n2Cc2ncc(Cl)cn2)cc1] | δ = 8.69 (s, 2H), 7.42 (d, 2H), 7.20 (d, 2H), 5.39 (s, 2H), 2.37 (s, 3H) |
| 1.016 | 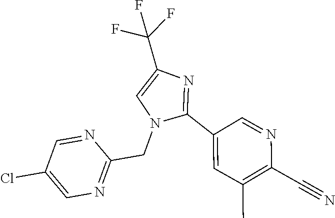 [SMILES: Cc1cc(-c2nc(C(F)(F)F)cn2Cc2ncc(Cl)cn2)cnc1C#N] | δ = 8.94 (d, 1H), 8.75 (s, 2H), 8.19 (d, 1H), 7.59 (d, 1H), 5.41 (s, 2H), 2.63 (s, 3H) |
| 1.017 | 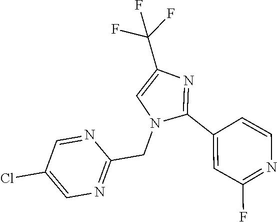 [SMILES: Fc1cc(-c2nc(C(F)(F)F)cn2Cc2ncc(Cl)cn2)ccn1] | δ = 8.74 (s, 2H), 8.33 (d, 1H), 7.61 (td, 1H), 7.57 (d, 1H), 7.42 (s, 1H), 5.44 (s, 2H) |
| 1.018 | 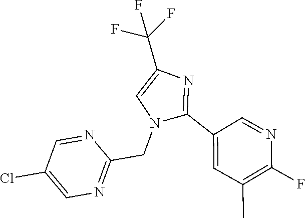 [SMILES: Cc1cc(-c2nc(C(F)(F)F)cn2Cc2ncc(Cl)cn2)cnc1F] | δ = 8.72 (s, 2H), 8.37 (d, 1H), 8.04 (m, 1H), 7.51 (q, 1H), 5.37 (s, 2H), 2.34 (s, 3H) |
| 1.019 | 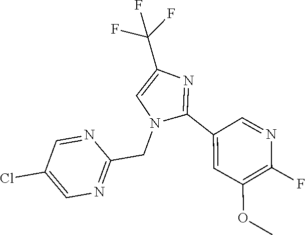 [SMILES: COc1cc(-c2nc(C(F)(F)F)cn2Cc2ncc(Cl)cn2)cnc1F] | δ = 8.74 (s, 2H), 8.07 (t, 1H), 7.77 (dd, 1H), 7.54 (q, 1H), 5.40 (s, 2H), 3.98 (s, 3H) |
| 1.020 | 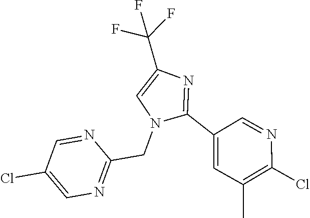 [SMILES: Cc1cc(-c2nc(C(F)(F)F)cn2Cc2ncc(Cl)cn2)cnc1Cl] | δ = 8.73 (s, 2H), 8.58 (dd, 1H), 8.02 (m, 1H), 7.54 (q, 1H), 5.38 (s, 2H), 2.45 (s, 3H) |
| 1.021 | 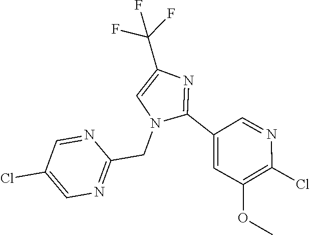 [SMILES: COc1cc(-c2nc(C(F)(F)F)cn2Cc2ncc(Cl)cn2)cnc1Cl] | δ = 8.73 (s, 2H), 8.32 (d, 1H), 7.70 (d, 1H), 7.55 (d, 1H), 5.40 (s, 2H), 4.00 (s, 3H) |
| 1.022 | 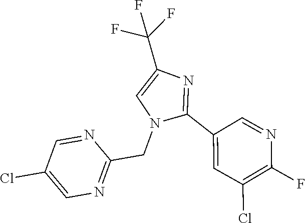 [SMILES: Fc1ncc(-c2nc(C(F)(F)F)cn2Cc2ncc(Cl)cn2)cc1Cl] | δ = 8.75 (s, 2H), 8.52 (dd, 1H), 8.35 (dd, 1H), 7.55 (q, 1H), 5.38 (s, 2H) |
| 1.023 | 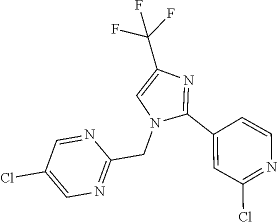 [SMILES: FC(F)(F)c1cn(Cc2ncc(Cl)cn2)c(-c2ccnc(Cl)c2)n1] | δ = 8.76 (s, 2H), 8.51 (dd, 1H), 7.83 (dd, 1H), 7.66 (dd, 1H), 7.58 (d, 1H), 5.44 (s, 2H) |
| 1.024 | 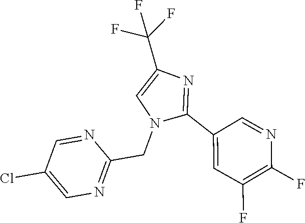 [SMILES: Fc1cc(-c2nc(C(F)(F)F)cn2Cc2ncc(Cl)cn2)cnc1F] | δ = 8.75 (s, 2H), 8.40 (t, 1H), 8.13 (dt, 1H), 7.54 (q, 1H), 5.38 (s, 2H) |
| 1.025 | 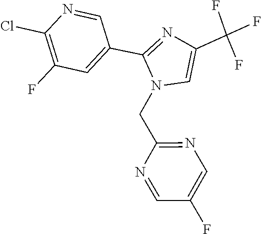 [SMILES: Fc1cnc(Cn2cc(C(F)(F)F)nc2-c2cnc(Cl)c(F)c2)nc1] | δ = 8.67-8.64 (m, 3H), 8.05 (dd, 1H), 7.57 (d, 1H), 5.41 (s, 2H) |
| 1.026 | 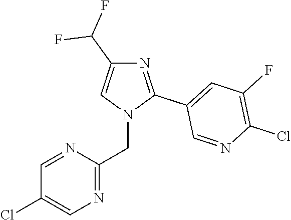 [SMILES: Fc1cc(-c2nc(C(F)F)cn2Cc2ncc(Cl)cn2)cnc1Cl] | MeOD: δ = 8.81 (s, 2H), 8.51 (d, 1H), 8.09 (dd, 1H), 7.69 (t, 1H), 6.77 (t, 1H), 5.56 (s, 2H) |
| 1.027 | 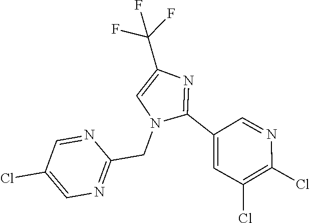 [SMILES: FC(F)(F)c1cn(Cc2ncc(Cl)cn2)c(-c2cnc(Cl)c(Cl)c2)n1] | δ = 8.74 (s, 2H), 8.71 (d, 1H), 8.31 (d, 1H), 7.55 (q, 1H), 5.38 (s, 2H) |
| 1.028 | 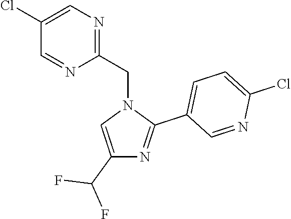 [SMILES: FC(F)c1cn(Cc2ncc(Cl)cn2)c(-c2ccc(Cl)nc2)n1] | δ ppm 8.72 (s, 2 H), 8.09 (dd, 1 H), 7.43-7.47 (m, 2 H), 7.27 (s, 1 H), 6.73 (t, 1 H), 5.37 (s, 2 H). |
| 1.029 | 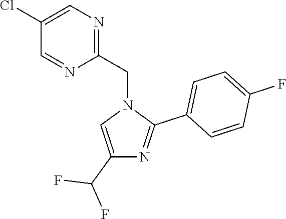 [SMILES: Fc1ccc(-c2nc(C(F)F)cn2Cc2ncc(Cl)cn2)cc1] | δ = 8.71 (s, 2H), 7.67 (m, 2H), 7.37 (t, 1H), 7.13 (t, 2H), 6.72 (t, 1H), 5.35 (s, 2H) |
| 1.030 | 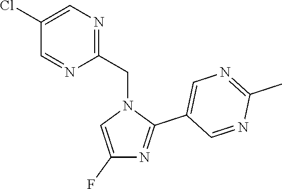 [SMILES: Cc1ncc(-c2nc(F)cn2Cc2ncc(Cl)cn2)cn1] | δ = 9.02 (s, 2H), 8.71 (s, 2H), 6.72 (d, 1H), 5.29 (s, 2H), 2.79 (s, 3H) |
| 1.031 | 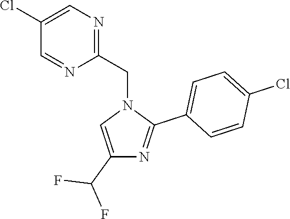 [SMILES: FC(F)c1cn(Cc2ncc(Cl)cn2)c(-c2ccc(Cl)cc2)n1] | δ = 8.71 (s, 2H), 7.63 (m, 2H), 7.42 (m, 2H), 7.38 (t, H), 6.71 (br t, 1H), 5.35 (s, 2H) |
| 1.032 | 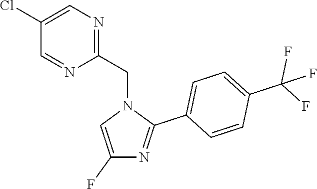 [SMILES: Fc1cn(Cc2ncc(Cl)cn2)c(-c2ccc(C(F)(F)F)cc2)n1] | δ = 8.71 (s, 2H), 7.81 (d, 2H), 7.68 (d, 2H), 6.68 (d, 1H), 5.31 (s, 2H) |
| 1.033 | 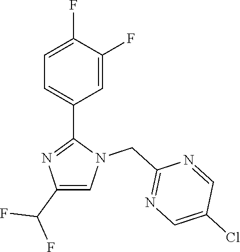 [SMILES: Fc1ccc(-c2nc(C(F)F)cn2Cc2ncc(Cl)cn2)cc1F] | δ = 8.72 (s, 2H), 7.66-7.59 (m, 1H), 7.49-7.43 (m, 1H), 7.37 (t, 1H), 7.28- 7.20 (m, 1H), 6.70 (t, 1H), 5.35 (s, 2H) |
| 1.034 | 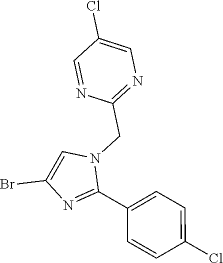 [SMILES: Clc1ccc(-c2nc(Br)cn2Cc2ncc(Cl)cn2)cc1] | δ = 8.70 (s, 2H), 7.64 (d, 2H), 7.40 (2, H), 7.09 (s, 1H), 5.30 (s, 2H) |
| 1.035 | 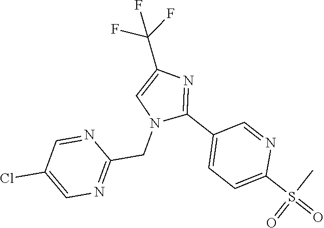 [SMILES: CS(=O)(=O)c1ccc(-c2nc(C(F)(F)F)cn2Cc2ncc(Cl)cn2)cn1] | δ = 9.15 (dd, 1H), 8.74 (s, 2H), 8.44 (dd, 1H), 8.19 (dd, 1H), 7.59 (q, 1H), 5.40 (s, 2H), 3.27 (s, 3H) |
| 1.036 | 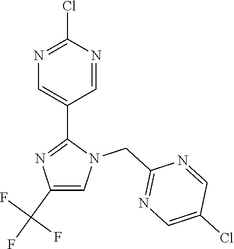 [SMILES: FC(F)(F)c1cn(Cc2ncc(Cl)cn2)c(-c2cnc(Cl)nc2)n1] | δ = 9.11 (s, 2H), 8.73 (s, 2H), 7.59 (q, 1H), 5.36 (s, 2H) |
| 1.037 | 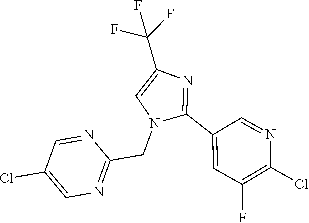 [SMILES: Fc1cc(-c2nc(C(F)(F)F)cn2Cc2ncc(Cl)cn2)cnc1Cl] | δ = 8.74 (s, 2H), 8.63 (d, 1H), 8.04 (dd, 1H), 7.55 (q, 1H), 5.39 (s, 2H) |
| 1.038 | 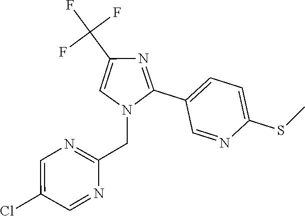 [SMILES: CSc1ccc(-c2nc(C(F)(F)F)cn2Cc2ncc(Cl)cn2)cn1] | δ = 8.73 (dd, 1H), 8.72 (s, 2H), 7.87 (dd, 1H), 7.49 (q, 1H), 7.28 (d, 1H), 5.37 (s, 2H), 2.59 (s, 3H) |
| 1.039 | 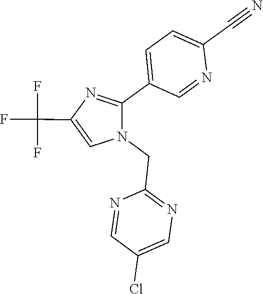 [SMILES: N#Cc1ccc(-c2nc(C(F)(F)F)cn2Cc2ncc(Cl)cn2)cn1] | δ = 9.16 (dd, 1H), 8.74 (s, 2H), 8.34 (dd, 1H), 7.81 (dd, 1H), 7.60 (q, 1H), 5.40 (s, 2H) |
| 1.040 | 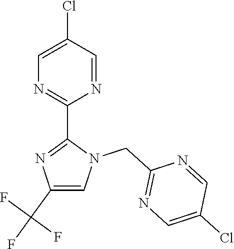 [SMILES: FC(F)(F)c1cn(Cc2ncc(Cl)cn2)c(-c2ncc(Cl)cn2)n1] | δ = 8.68 (s, 2H), 8.59 (s, 2H), 7.51 (m, 1H), 6.05 (s, 2H) |
| 1.041 | 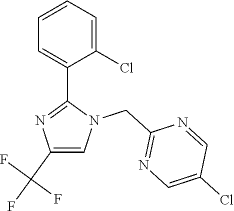 [SMILES: FC(F)(F)c1cn(Cc2ncc(Cl)cn2)c(-c2ccccc2Cl)n1] | δ = 8.61 (s, 2H), 7.52 (d, 1H), 7.49- 7.37 (m, 3H), 7.30 (dt, 1H), 5.22 (s, 2H) |
| 1.042 | 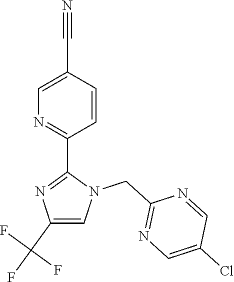 [SMILES: N#Cc1ccc(-c2nc(C(F)(F)F)cn2Cc2ncc(Cl)cn2)nc1] | δ = 8.60 (dd, 1H), 8.59 (s, 2H), 8.44 (dd, 1H), 7.99 (dd, 1H), 7.49 (m, 1H), 6.10 (s, 2H) |
| 1.043 | 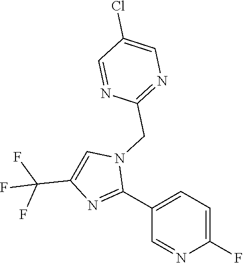 [SMILES: Fc1ccc(-c2nc(C(F)(F)F)cn2Cc2ncc(Cl)cn2)cn1] | δ = 8.73 (s, 2H), 8.60 (d, 1H), 8.22 (ddd, 1H), 7.53 (d, 1H), 7.06 (ddd, 1H), 5.36 (s, 2H) |
| 1.044 | 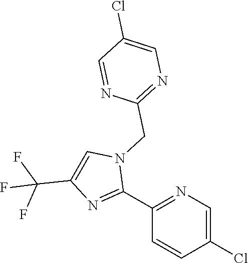 [SMILES: FC(F)(F)c1cn(Cc2ncc(Cl)cn2)c(-c2ccc(Cl)cn2)n1] | δ = 8.59 (s, 2H), 8.30 (dd, 1H), 8.24 (dd, 1H), 7.71 (dd, 1H), 7.43 (m, 1H), 6.06 (s, 2H) |
| 1.045 | 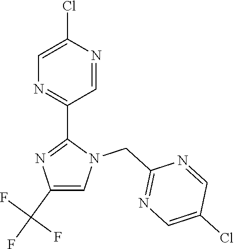 [SMILES: FC(F)(F)c1cn(Cc2ncc(Cl)cn2)c(-c2cnc(Cl)cn2)n1] | δ = 9.31 (d, 1H), 8.60 (s, 2H), 8.33 (d, 1H), 7.49 (q, 1H), 6.01 (s, 2H) |
| 1.046 | 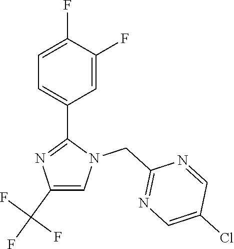 [SMILES: Fc1ccc(-c2nc(C(F)(F)F)cn2Cc2ncc(Cl)cn2)cc1F] | δ = 8.73 (s, 2H), 7.64 (ddd, 1H), 7.48 (m, 2H), 7.25 (m, 1H), 5.36 (s, 2H) |
| 1.047 | 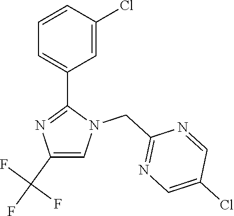 [SMILES: FC(F)(F)c1cn(Cc2ncc(Cl)cn2)c(-c2cccc(Cl)c2)n1] | δ = 8.73 (s, 2H), 7.74 (t, 1H), 7.57 (dt, 1H), 7.49 (m, 1H), 7.44 (ddd, 1H), 7.38 (t, 1H), 5.38 (s, 2H) |
| 1.048 | 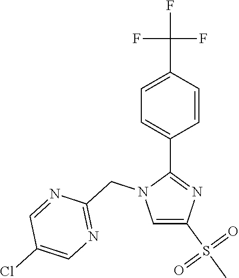 [SMILES: CS(=O)(=O)c1cn(Cc2ncc(Cl)cn2)c(-c2ccc(C(F)(F)F)cc2)n1] | δ = 8.73 (s, 2H), 7.87 (d, 2H), 7.84 (s, 1H), 7.75 (d, 2H), 5.41 (s, 2H), 3.23 (s, 3H) |
| 1.049 | 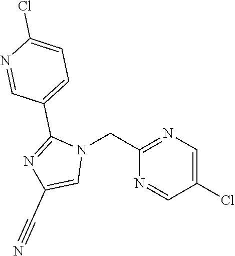 [SMILES: N#Cc1cn(Cc2ncc(Cl)cn2)c(-c2ccc(Cl)nc2)n1] | δ = 8.70-8.74 (m, 3 H), 8.04 (dd, 1 H), 7.75 (s, 1 H), 7.46 (d, 1 H), 5.38 (s, 2 H) |
| 1.050 | 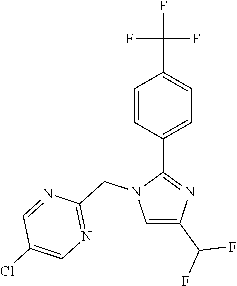 [SMILES: FC(F)c1cn(Cc2ncc(Cl)cn2)c(-c2ccc(C(F)(F)F)cc2)n1] | δ = 8.72 (s, 2H), 7.85 (d, 2H), 7.71 (d, 2H), 7.42 (t, 1H), 6.73 (t, 1H), 5.38 (s, 2H) |
| 1.051 | 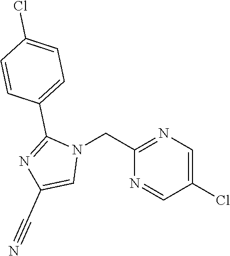 [SMILES: N#Cc1cn(Cc2ncc(Cl)cn2)c(-c2ccc(Cl)cc2)n1] | δ ppm 8.71 (s, 2 H), 7.70 (s, 1 H), 7.59 (d, 2 H), 7.43 (d, 2 H), 5.37 (s, 2 H) |
| 1.052 | 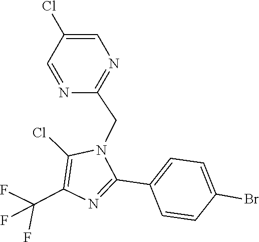 [SMILES: FC(F)(F)c1nc(-c2ccc(Br)cc2)n(Cc2ncc(Cl)cn2)c1Cl] | δ = 8.71 (s, 2H), 7.56 (d, 2H), 7.45 (d, 2H), 5.39 (s, 2H) |
| 1.053 | 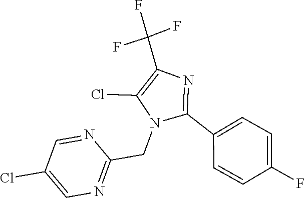 [SMILES: Fc1ccc(-c2nc(C(F)(F)F)c(Cl)n2Cc2ncc(Cl)cn2)cc1] | δ = 8.71 (s, 2H), 7.61-7.52 (m, 2H), 7.11 (t, 2H), 5.38 (s, 2H) |
| 1.054 | 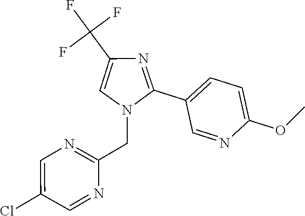 [SMILES: COc1ccc(-c2nc(C(F)(F)F)cn2Cc2ncc(Cl)cn2)cn1] | δ = 8.73 (s, 2H), 8.47 (dd, 1H), 7.94 (dd, 1H), 7.48 (d, 1H), 6.84 (dd, 1H), 5.36 (s, 2H), 3.98 (s, 3H) |
| 1.055 | 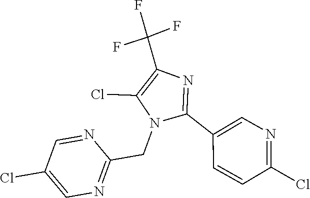 [SMILES: FC(F)(F)c1nc(-c2ccc(Cl)nc2)n(Cc2ncc(Cl)cn2)c1Cl] | δ = 8.70 (s, 2H), 8.60 (dd, 1H), 7.97 (dd, 1H), 7.42 (dd, 1H), 5.39 (s, 2H) |
| 1.056 | 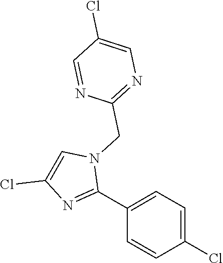 [SMILES: Clc1ccc(-c2nc(Cl)cn2Cc2ncc(Cl)cn2)cc1] | δ = 8.71 (s, 2H), 7.61 (d, 2H), 7.39 (d, 2H), 7.01 (s, 1H), 5.30 (s, 2H) |
| 1.057 | 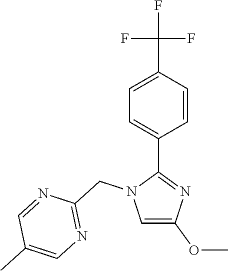 | δ = 8.59 (s, 2H), 7.92 (d, 2H), 7.66 (d, 2H), 6.47 (s, 1H), 5.28 (s, 2H), 3.84 (s, 3H), 2.35 (s, 3H) |
| 1.058 | 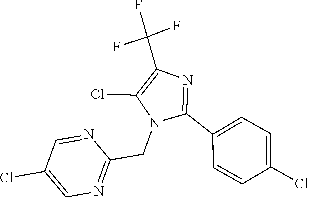 [SMILES: FC(F)(F)c1nc(-c2ccc(Cl)cc2)n(Cc2ncc(Cl)cn2)c1Cl] | δ = 8.71 (s, 2H), 7.52 (d, 2H), 7.40 (d, 2H), 5.39 (s, 2H) |
| 1.059 | 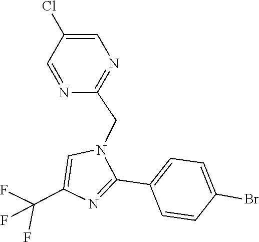 [SMILES: FC(F)(F)c1cn(Cc2ncc(Cl)cn2)c(-c2ccc(Br)cc2)n1] | δ = 8.72 (s, 2 H), 7.62-7.51 (m, 4H), 7.49 (s, 1H), 5.36 (s, 2H) |
| 1.060 | 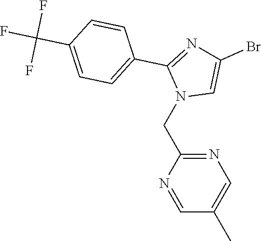 [SMILES: Cc1cnc(Cn2cc(Br)nc2-c2ccc(C(F)(F)F)cc2)nc1] | δ = 8.58 (s, 2H), 7.90 (d, 2H), 7.69 (d, 2H), 7.16 (s, 1H), 5.31 (s, 2H), 2.35 (s, 3H) |
| 1.061 | 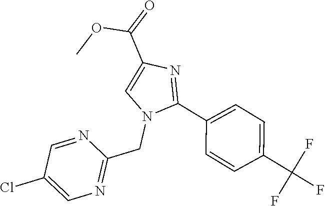 [SMILES: COC(=O)c1cn(Cc2ncc(Cl)cn2)c(-c2ccc(C(F)(F)F)cc2)n1] | δ = 8.70 (s, 2H), 7.86 (s, 1H), 7.84 (d, 2H), 7.69 (d, 2H), 5.39 (s, 2H), 3.91 (s, 3H) |
| 1.062 | 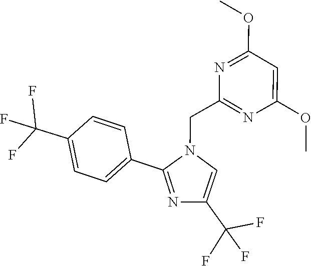 [SMILES: COc1cc(OC)nc(Cn2cc(C(F)(F)F)nc2-c2ccc(C(F)(F)F)cc2)n1] | δ = 7.91 (d, 2H), 7.71 (d, 2H), 7.54 (s, 1H), 5.99 (s, 1H), 5.20 (s, 2H), 3.88 (s, 6H) |
| 1.063 | 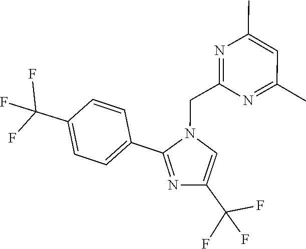 [SMILES: Cc1cc(C)nc(Cn2cc(C(F)(F)F)nc2-c2ccc(C(F)(F)F)cc2)n1] | δ = 7.96 (d, 2H), 7.71 (d, 2H), 7.53 (s, 1H), 7.00 (s, 1H), 5.28 (s, 2H), 2.48 (s, 6H) |
| 1.064 | 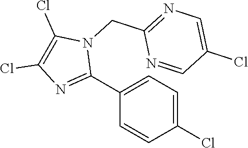 [SMILES: Clc1ccc(-c2nc(Cl)c(Cl)n2Cc2ncc(Cl)cn2)cc1] | δ = 8.69 (s, 2H), 7.55-7.48 (m, 2H), 7.41-7.33 (m, 2H), 5.36 (s, 2H) |
| 1.065 | 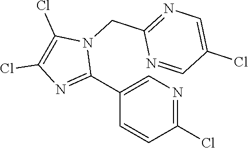 [SMILES: Clc1cnc(Cn2c(-c3ccc(Cl)nc3)nc(Cl)c2Cl)nc1] | δ = 8.70 (s, 2H), 8.60 (d, 1H), 7.96 (dd, 1H), 7.41 (d, 1H), 5.38 (s, 2H) |
| 1.066 | 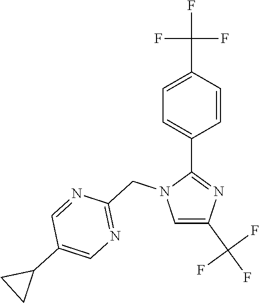 [SMILES: FC(F)(F)c1ccc(-c2nc(C(F)(F)F)cn2Cc2ncc(C3CC3)cn2)cc1] | δ = 8.47 (s, 2H), 7.91 (d, 2H), 7.71 (d, 2H), 7.56-7.50 (m, 1H), 5.33 (s, 2H), 1.93-1.84 (m, 1H), 1.18-1.10 (m, 2H), 0.86-0.78 (m, 2H) |
| 1.067 | 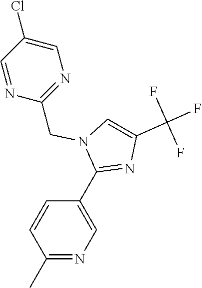 [SMILES: Cc1ccc(-c2nc(C(F)(F)F)cn2Cc2ncc(Cl)cn2)cn1] | δ = 8.79 (s, 1H), 8.71 (s, 2H), 7.95 (dd, 1H), 7.51 (d, 1H), 7.26 (d, 1H), 5.36 (s, 2H), 2.61 (s, 3H) |
| 1.068 | 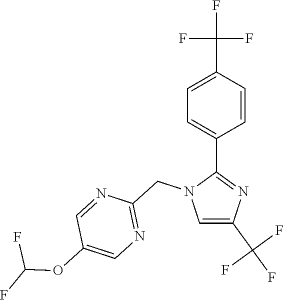 [SMILES: FC(F)Oc1cnc(Cn2cc(C(F)(F)F)nc2-c2ccc(C(F)(F)F)cc2)nc1] | δ = 8.64 (s, 2H), 7.88 (d, 2H), 7.72 (d, 2H), 7.55-7.53 (m, 1H), 6.64 (t, 1H), 5.40 (s, 2H) |
| 1.069 | 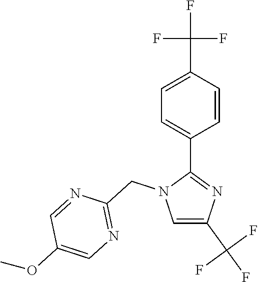 [SMILES: COc1cnc(Cn2cc(C(F)(F)F)nc2-c2ccc(C(F)(F)F)cc2)nc1] | δ = 8.41 (s, 2H), 7.93 (d, 2H), 7.72 (d, 2H), 7.56-7.52 (m, 1H), 5.33 (s, 2H), 3.95 (s, 3H) |
| 1.070 | 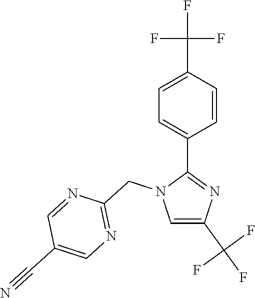 [SMILES: N#Cc1cnc(Cn2cc(C(F)(F)F)nc2-c2ccc(C(F)(F)F)cc2)nc1] | δ = 9.02 (s, 2H), 7.79 (d, 2H), 7.72 (d, 2H), 7.52-7.48 (m, 1H), 5.49 (s, 2H) |
| 1.071 | 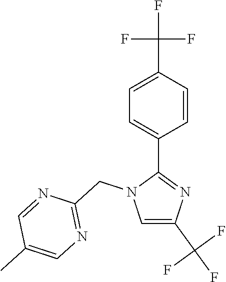 [SMILES: Cc1cnc(Cn2cc(C(F)(F)F)nc2-c2ccc(C(F)(F)F)cc2)nc1] | δ = 8.59 (s, 2H), 7.91 (d, 2H), 7.71 (d, 2H), 7.56-7.52 (m, 1H), 5.35 (s, 2H), 2.35 (s, 3H) |
| 1.072 | 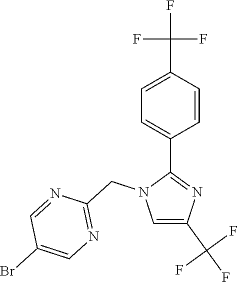 [SMILES: FC(F)(F)c1ccc(-c2nc(C(F)(F)F)cn2Cc2ncc(Br)cn2)cc1] | δ = 8.82 (s, 2H), 7.86 (d, 2H), 7.72 (d, 2H), 7.53-7.50 (m, 1H), 5.36 (s, 2H) |
| 1.073 | 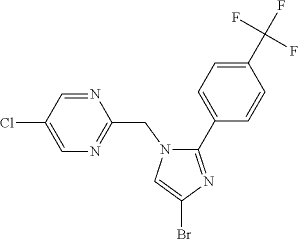 [SMILES: FC(F)(F)c1ccc(-c2nc(Br)cn2Cc2ncc(Cl)cn2)cc1] | δ = 8.72 (s, 2H), 7.85 (d, 2H), 7.70 (d, 2H), 7.15 (s, 1H), 5.34 (s, 2H) |
| 1.074 | 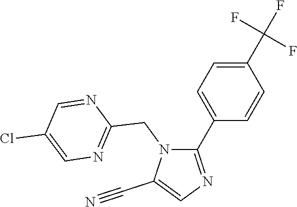 [SMILES: N#Cc1cnc(-c2ccc(C(F)(F)F)cc2)n1Cc1ncc(Cl)cn1] | δ = 8.71 (s, 2H), 7.83 (s, 1H), 7.73 (d, 2H), 7.71 (d, 2H), 5.53 (s, 2H) |
| 1.075 | 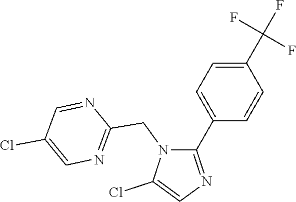 [SMILES: FC(F)(F)c1ccc(-c2ncc(Cl)n2Cc2ncc(Cl)cn2)cc1] | δ = 8.70 (s, 2H), 7.72 (d, 2H), 7.66 (d, 2H), 7.17 (s, 1H), 5.41 (s, 2H) |
| 1.076 | 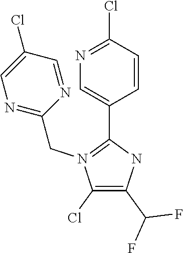 | 1H NMR (400 MHz, METHANOL-d4) δ ppm 8.72 (s, 2 H), 8.51 (dd, 1 H), 7.94 (dd, 1 H), 7.45 (dd, 1 H), 6.72 (t,1 H), 5.43 (s, 2 H) |
| 1.077 | 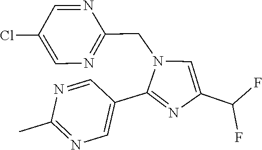 [SMILES: Cc1ncc(-c2nc(C(F)F)cn2Cc2ncc(Cl)cn2)cn1] | 1H NMR (400 MHz, CHLOROFORM-d) δ ppm 9.02 (m, 2 H), 8.70 (s, 2 H), 7.45 (s, 1 H), 6.71 (t, 1 H), 5.34 (s, 2 H), 2.77-2.81 (m, 3 H) |
| 1.078 | 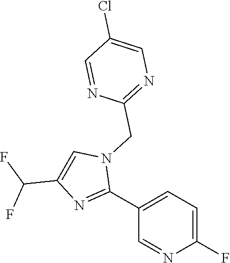 [SMILES: Fc1ccc(-c2nc(C(F)F)cn2Cc2ncc(Cl)cn2)cn1] | 1H NMR (400 MHz, CHLOROFORM-d) δ ppm 8.72 (s, 2 H) 8.60 (s, 1 H) 8.18- 8.25 (m, 1 H) 7.43 (s, 1 H) 7.05 (dd, 1 H) 6.72 (t, 1 H) 5.36 (s, 2 H) |
| 1.079 | 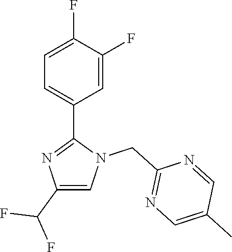 [SMILES: Cc1cnc(Cn2cc(C(F)F)nc2-c2ccc(F)c(F)c2)nc1] | 1H NMR (400 MHz, CHLOROFORM-d) δ ppm 8.60 (s, 2 H), 7.69 (ddd, 1 H), 7.48-7.55 (m, 1 H), 7.39 (t, 1 H), 7.24 (d, 1 H), 6.71 (t, 1 H), 5.33 (s, 2 H), 2.36 (s, 3 H) |
| 1.080 | 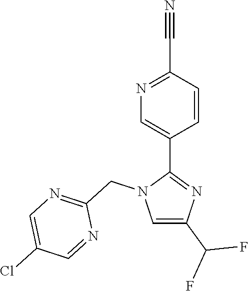 [SMILES: N#Cc1ccc(-c2nc(C(F)F)cn2Cc2ncc(Cl)cn2)cn1] | 1H NMR (400 MHz, CHLOROFORM-d) δ ppm 9.12 (d, 1 H), 8.71 (s, 2 H), 8.31 (dd, 1 H), 7.78 (d, 1 H), 7.48 (s, 1 H), 6.69 (t, 1H), 5.39 (s, 2 H), |
| 1.081 | 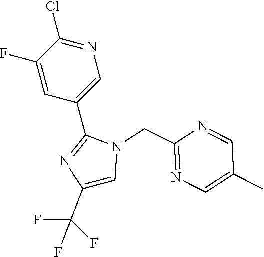 [SMILES: Cc1cnc(Cn2cc(C(F)(F)F)nc2-c2cnc(Cl)c(F)c2)nc1] | 1H NMR (400 MHz, chloroform) δ = 8.70 (d, 1H), 8.60 (s, 2H), 8.11 (dd, 1H), 7.57 (d, 1H), 5.36 (s, 2H), 2.37 (s, 3H) |
| 1.082 | 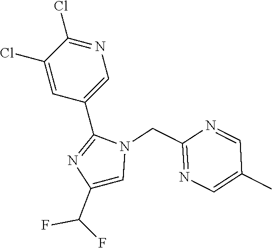 [SMILES: Cc1cnc(Cn2cc(C(F)F)nc2-c2cnc(Cl)c(Cl)c2)nc1] | 1H NMR (400 MHz, CHLOROFORM-d) δ ppm 8.75 (d, 1 H), 8.58 (s, 2 H), 8.35 (d, 1 H), 7.46 (t, 1 H), 6.70 (t, 1 H), 5.33 (s, 2 H), 2.35 (s, 3 H) |
| 1.083 | 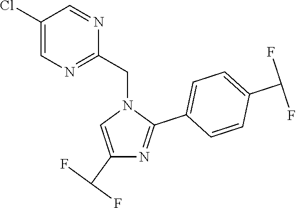 [SMILES: FC(F)c1ccc(-c2nc(C(F)F)cn2Cc2ncc(Cl)cn2)cc1] | 1H NMR (400 MHz, CHLOROFORM-d) δ ppm 8.72 (s, 2 H) 7.80 (d, 2 H) 7.60 (d, 2 H) 7.41 (t, 1 H) 6.88 (s, 1 H) 6.84 (s, 1 H) 6.74 (s, 1 H) 6.69 (s, 1 H) 6.60 (s, 1 H) 6.55 (s, 1 H) 5.38 (s, 2 H) |
| 1.084 | 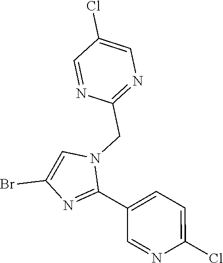 [SMILES: Clc1cnc(Cn2cc(Br)nc2-c2ccc(Cl)nc2)nc1] | 1H NMR (400 MHz, DMSO-d6) δ ppm 8.77 (d, 1 H), 8.72 (s, 2 H), 8.09 (dd, 1 H), 7.43 (d, 1 H), 7.17 (s, 1 H), 5.32 (s, 2 H) |
| 1.085 | 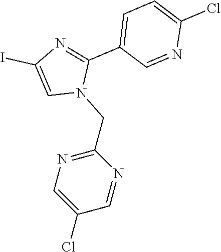 [SMILES: Clc1cnc(Cn2cc(I)nc2-c2ccc(Cl)nc2)nc1] | 1H NMR (400 MHz, DMSO-d6) δ ppm 8.92 (s, 2 H) 8.57 (d, 1 H) 8.01 (dd, 1 H) 7.62 (s, 1 H) 7.56 (d, 1 H) 5.58 (s, 2H) |
| 1.086 | 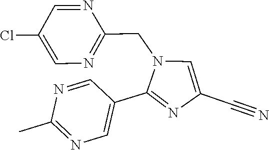 [SMILES: Cc1ncc(-c2nc(C#N)cn2Cc2ncc(Cl)cn2)cn1] | 1H NMR (400 MHz, DMSO-d6) δ ppm 8.99 (s, 2 H), 8.71 (s, 2 H), 7.77 (s, 1 H), 5.37 (s, 2 H), 2.80 (s, 3 H) |
| 1.087 | 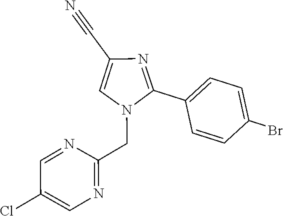 [SMILES: N#Cc1cn(Cc2ncc(Cl)cn2)c(-c2ccc(Br)cc2)n1] | 1H NMR (400 MHz, DMSO-d6) δ ppm 8.72 (s, 2 H) 7.71 (s, 1 H) 7.61 (m, 2 H) 7.54 (m, 2 H) 5.38 (s, 2 H) |
| 1.088 | 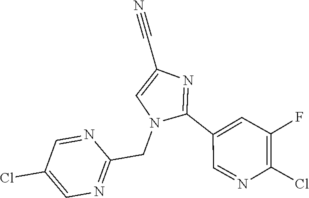 [SMILES: N#Cc1cn(Cc2ncc(Cl)cn2)c(-c2cnc(Cl)c(F)c2)n1] | 1H NMR (400 MHz, DMSO-d6) δ ppm 8.75 (s, 2 H) 8.60 (d, 1 H) 8.00 (dd, 1 H) 7.77 (s, 1 H) 5.41 (s, 2 H) |
| 1.089 | 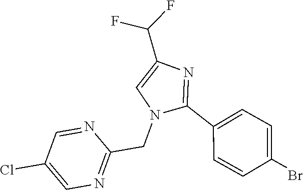 [SMILES: FC(F)c1cn(Cc2ncc(Cl)cn2)c(-c2ccc(Br)cc2)n1] | 1H NMR (400 MHz, DMSO-d6) δ ppm 8.72 (s, 2 H) 7.58 (s, 4 H) 7.39 (t, 1 H) 6.86 (s, 1 H) 6.72 (s, 1 H) 6.58 (s, 1 H) 5.36(s, 2 H) |
| 1.090 | 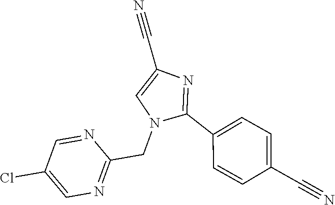 [SMILES: N#Cc1ccc(-c2nc(C#N)cn2Cc2ncc(Cl)cn2)cc1] | 1H NMR (400 MHz, DMSO-d6) δ ppm 8.74 (s, 2 H) 7.83-7.87 (m, 2 H) 7.77- 7.79 (m, 1 H) 7.75-7.80 (m, 2 H) 5.41 (s, 2 H) |
| 1.091 | 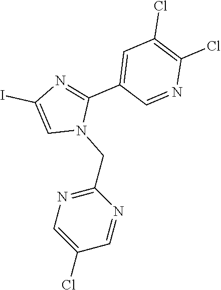 [SMILES: Clc1cnc(Cn2cc(I)nc2-c2cnc(Cl)c(Cl)c2)nc1] | 1H NMR (400 MHz, DMSO-d6) δ ppm 8.72 (s, 2 H), 8.69 (d, 1 H), 8.29 (d, 1 H), 7.27 (d, 1 H), 5.33 (s, 2 H) |
| 1.092 | 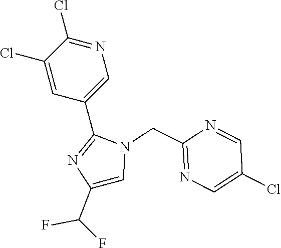 [SMILES: FC(F)c1cn(Cc2ncc(Cl)cn2)c(-c2cnc(Cl)c(Cl)c2)n1] | ¹H NMR (400 MHz, CHLOROFORM-d) δ ppm 8.72 (s, 2 H), 8.69 (d, 1 H), 8.29 (d, 1 H), 7.44 (t, 1 H), 6.69 (s, 1 H), 5.37 (s, 2 H) |
| 1.093 | 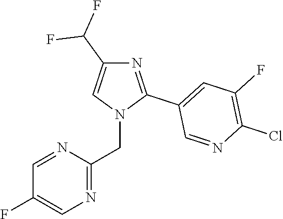 [SMILES: Fc1cnc(Cn2cc(C(F)F)nc2-c2cnc(Cl)c(F)c2)nc1] | 1H NMR (400 MHz, CHLOROFORM-d) δ ppm 8.63-8.65 (m, 3 H) 8.03 (dd, 1 H) 7.46 (s, 1 H) 6.55-6.83 (m, 1 H) 5.40 (s, 2 H) |
| 1.094 | 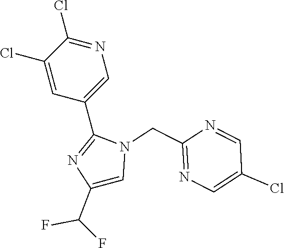 [SMILES: FC(F)c1cn(Cc2ncc(Cl)cn2)c(-c2cnc(Cl)c(Cl)c2)n1] | 1H NMR (400 MHz, CHLOROFORM-d) δ ppm 8.73 (s, 2 H), 8.69 (d, 1 H), 8.30 (d, 1 H), 7.45 (t, 1 H), 6.70 (t, 1 H), 5.37 (s, 2 H) |
| 1.095 | 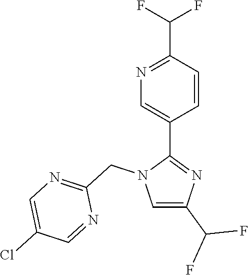 [SMILES: FC(F)c1ccc(-c2nc(C(F)F)cn2Cc2ncc(Cl)cn2)cn1] | 1H NMR (400 MHz, CHLOROFORM-d) δ ppm 9.04 (s, 1 H) 8.73 (s, 2 H) 8.28 (dd, 1 H) 7.76 (d, 1 H) 7.47 (s, 1 H) 6.74 (t, 1 H) 6.69 (t, 1 H) 5.39 (s, 2 H) |
| 1.096 | 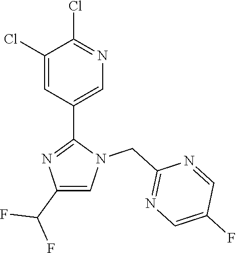 [SMILES: Fc1cnc(Cn2cc(C(F)F)nc2-c2cnc(Cl)c(Cl)c2)nc1] | 1H NMR (400 MHz, CHLOROFORM-d) δ ppm 8.73 (d, 1 H), 8.65 (s, 2 H), 8.32 (d, 1 H), 7.47 (t, 1 H), 6.71 (t, 1 H), 5.40 (s, 2 H), |
| 1.097 | 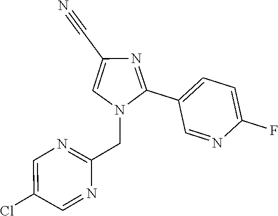 [SMILES: N#Cc1cn(Cc2ncc(Cl)cn2)c(-c2ccc(F)nc2)n1] | 1H NMR (400 MHz, CHLOROFORM-d) δ ppm 8.73 (s, 2 H), 8.57 (d, 1 H), 8.19 (m, 1 H), 7.75 (s, 1 H), 7.08 (dd, 1 H), 5.38 (s, 2 H). |
| 1.098 | 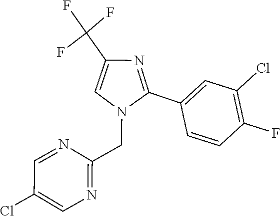 [SMILES: Fc1ccc(-c2nc(C(F)(F)F)cn2Cc2ncc(Cl)cn2)cc1Cl] | 1H NMR (400 MHz, chloroform) δ = 8.74 (s, 2H), 7.85 (dd, 1H), 7.61 (m, 1H), 7.49 (m, 1H), 7.23 (t, 1H), 5.36 (s, 2H) |
| 1.099 | 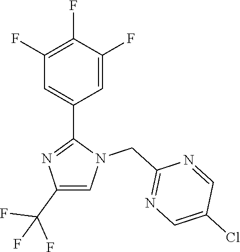 [SMILES: Fc1cc(-c2nc(C(F)(F)F)cn2Cc2ncc(Cl)cn2)cc(F)c1F] | 1H NMR (400 MHz, chloroform) δ = 8.76 (s, 2H), 7.50 (m, 3H), 5.38 (s, 2H) |
| 1.100 | 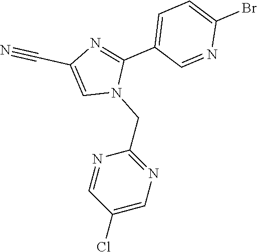 [SMILES: N#Cc1cn(Cc2ncc(Cl)cn2)c(-c2ccc(Br)nc2)n1] | 1H NMR (400 MHz, CHLOROFORM-d) δ ppm 8.73 (s, 2 H), 8.71 (d, 1 H), 7.95 (dd, 1 H), 7.76 (s, 1 H), 7.63 (d, 1H), 5.38 (s, 2 H) |
| 1.101 | 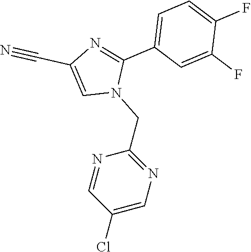 [SMILES: N#Cc1cn(Cc2ncc(Cl)cn2)c(-c2ccc(F)c(F)c2)n1] | 1H NMR (400 MHz, CHLOROFORM-d) δ ppm 8.74 (s, 1 H), 8.71-8.73 (m, 1 H), 7.71 (s, 1 H), 7.56-7.64 (m, 1 H), 7.41-7.47 (m, 1 H), 7.23-7.31 (m, 1 H), 5.38 (s, 2 H). |
| 1.102 | 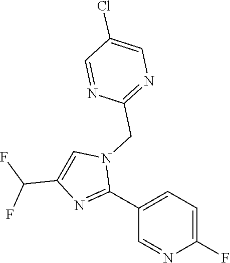 [SMILES: Fc1ccc(-c2nc(C(F)F)cn2Cc2ncc(Cl)cn2)cn1] | 1H NMR (400 MHz, CHLOROFORM-d) δ ppm 8.72 (s, 2 H), 8.60 (d, 1 H), 8.22 (m, 1 H), 7.43 (t, 1 H), 7.05 (dd, 1 H), , 6.72 (t, 1 H), 5.36 (s, 2 H). |
| 1.103 | 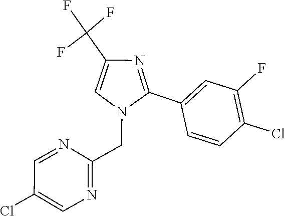 [SMILES: Fc1cc(-c2nc(C(F)(F)F)cn2Cc2ncc(Cl)cn2)ccc1Cl] | 1H NMR (400 MHz, chloroform) δ = 8.74 (s, 2H), 7.62 (m, 1H), 7.49 (m, 3H), 5.38 (s, 2H) |
| 1.104 | 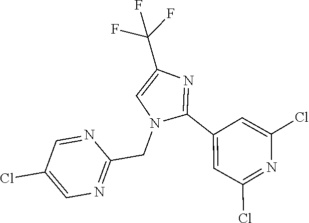 [SMILES: FC(F)(F)c1cn(Cc2ncc(Cl)cn2)c(-c2cc(Cl)nc(Cl)c2)n1] | 1H NMR (400 MHz, chloroform) δ = 8.77 (s, 2H), 7.80 (s, 2H), 7.59 (d, 1H), 5.44 (s, 2H) |

### BIOLOGICAL EXAMPLES

Seeds of a variety of test species are sown in standard soil in pots Amaranthus palmeri (AMAPA), Amaranthus retroflexus (AMARE), Setaria faberi (SETFA), Echinochloa crus-galli (ECHCG), Ipomoea hederacea (IPOHE)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethylene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| | Rate | | | | | |
|---|---|---|---|---|---|---|
| Compound | (g/ha) | AMAPA | AMARE | SETFA | ECHCG | IPOHE |
| 1.001 | 250 | 5 | 5 | 5 | 5 | 5 |
| 1.002 | 250 | 5 | 5 | 5 | 5 | 5 |
| 1.003 | 250 | 5 | 5 | 5 | 5 | 4 |
| 1.004 | 250 | 5 | 5 | 5 | 5 | 5 |
| 1.005 | 250 | 5 | 5 | 5 | 5 | 5 |
| 1.006 | 250 | 5 | 5 | 5 | 5 | 4 |
| 1.007 | 250 | 5 | 5 | 5 | 5 | 5 |
| 1.008 | 250 | 5 | 5 | 5 | 5 | 5 |
| 1.009 | 250 | 5 | 5 | 4 | 4 | 4 |
| 1.010 | 250 | 4 | 4 | 4 | 4 | 4 |
| 1.011 | 250 | 5 | 5 | 5 | 5 | 4 |
| 1.012 | 250 | 5 | 5 | 5 | 5 | 3 |
| 1.013 | 250 | 5 | 5 | 5 | 5 | 5 |
| 1.014 | 250 | 5 | 5 | 5 | 5 | 3 |
| 1.015 | 250 | 5 | 5 | 5 | 5 | 3 |
| 1.016 | 250 | 5 | 5 | NT | 5 | 4 |
| 1.017 | 250 | 5 | 5 | NT | 5 | 4 |
| 1.018 | 250 | 5 | 5 | NT | 5 | 4 |
| 1.019 | 250 | 5 | 5 | NT | 5 | 4 |
| 1.020 | 250 | 5 | 5 | NT | 5 | 4 |
| 1.021 | 250 | 5 | 5 | NT | 4 | 4 |
| 1.022 | 250 | 5 | 5 | NT | 5 | 4 |
| 1.023 | 250 | 4 | 5 | NT | 5 | 4 |
| 1.024 | 250 | 4 | 4 | NT | 3 | 2 |
| 1.025 | 250 | 5 | 5 | NT | 4 | 4 |
| 1.026 | 250 | 4 | 4 | NT | 4 | 5 |
| 1.027 | 250 | 4 | 4 | NT | 4 | 4 |
| 1.028 | 250 | 4 | 4 | NT | 4 | 4 |
| 1.029 | 250 | 5 | 5 | NT | 4 | 5 |
| 1.030 | 250 | 2 | 3 | NT | 1 | 3 |
| 1.031 | 250 | 4 | 4 | NT | 4 | 5 |
| 1.032 | 250 | 3 | 3 | NT | 4 | 4 |
| 1.033 | 250 | 4 | 4 | NT | 5 | 5 |
| 1.034 | 250 | 3 | 4 | 3 | 3 | 2 |
| 1.035 | 250 | 5 | 5 | 5 | 4 | 4 |
| 1.036 | 250 | 1 | 1 | 1 | 1 | 1 |
| 1.037 | 250 | 5 | 5 | 5 | 4 | 4 |
| 1.038 | 250 | 5 | 5 | 5 | 5 | 4 |
| 1.039 | 250 | 5 | 5 | 5 | 5 | 4 |
| 1.040 | 250 | 1 | 1 | 1 | 1 | 1 |
| 1.041 | 250 | 3 | 2 | 1 | 1 | 3 |
| 1.042 | 250 | 3 | 3 | NT | 1 | 2 |
| 1.043 | 250 | 5 | 5 | NT | 4 | 5 |
| 1.044 | 250 | 3 | 3 | NT | 1 | 2 |
| 1.045 | 250 | 1 | 1 | NT | 1 | 2 |
| 1.046 | 250 | 5 | 5 | NT | 5 | 5 |
| 1.047 | 250 | 5 | 5 | 5 | 4 | 4 |
| 1.048 | 250 | 4 | 5 | 4 | 4 | 4 |
| 1.019 | 250 | 4 | 5 | 3 | 3 | 3 |
| 1.050 | 250 | NT | 5 | 5 | 5 | 4 |
| 1.051 | 250 | NT | 5 | 4 | 4 | 3 |
| 1.052 | 250 | 5 | 5 | 4 | 4 | 4 |
| 1.053 | 250 | 5 | 5 | 5 | 5 | 3 |
| 1.054 | 250 | 5 | 5 | 5 | 5 | 3 |
| 1.055 | 250 | 5 | 5 | 5 | 5 | 4 |
| 1.056 | 250 | 4 | 4 | 3 | 3 | 2 |
| 1.057 | 250 | 1 | 1 | 1 | 1 | 2 |
| 1.058 | 250 | 5 | 5 | 5 | 5 | 4 |
| 1.059 | 250 | 5 | 5 | 5 | 4 | 3 |
| 1.060 | 250 | 5 | 5 | 5 | 5 | 3 |
| 1.061 | 250 | 4 | 4 | 1 | 1 | 1 |
| 1.062 | 250 | 1 | 1 | NT | 1 | 1 |
| 1.063 | 250 | 1 | 1 | NT | 1 | 1 |
| 1.064 | 250 | 5 | 5 | 3 | 2 | 3 |
| 1.065 | 250 | NT | 5 | 4 | 3 | 3 |
| 1.066 | 250 | 5 | 5 | 5 | 4 | 4 |
| 1.067 | 250 | 5 | 5 | 5 | 5 | 4 |
| 1.068 | 250 | 5 | 5 | 4 | 4 | 3 |
| 1.069 | 250 | 5 | 5 | 5 | 5 | 4 |
| 1.070 | 250 | 5 | 5 | 5 | 5 | 4 |
| 1.071 | 250 | 5 | 5 | 5 | 5 | 4 |
| 1.072 | 250 | 5 | 5 | 5 | 5 | 4 |
| 1.073 | 250 | 5 | 5 | 5 | 4 | 4 |
| 1.074 | 250 | 2 | 1 | 1 | 1 | 2 |
| 1.075 | 250 | 3 | 3 | 2 | 3 | 3 |

NT = Not tested

TABLE B2
Pre-emergence Test

| | Rate | | | | | |
|---|---|---|---|---|---|---|
| Compound | (g/ha) | AMAPA | AMARE | SETFA | ECHCG | IPOHE |
| 1.001 | 250 | 5 | 5 | 5 | 5 | 5 |
| 1.002 | 250 | 5 | 5 | 5 | 5 | 5 |
| 1.003 | 250 | 5 | 5 | 5 | 5 | 5 |
| 1.004 | 250 | 5 | 5 | 5 | 5 | 5 |
| 1.005 | 250 | 5 | 5 | 5 | 5 | 5 |
| 1.006 | 250 | 5 | 5 | 5 | 5 | 5 |
| 1.007 | 250 | 5 | 5 | 5 | 5 | 5 |
| 1.008 | 250 | 5 | 5 | 5 | 5 | 5 |
| 1.009 | 250 | 5 | 5 | 5 | 5 | 5 |
| 1.010 | 250 | 5 | 5 | 5 | 5 | 2 |
| 1.011 | 250 | 5 | 5 | 5 | 5 | 5 |
| 1.012 | 250 | 4 | 5 | 5 | 5 | 5 |
| 1.013 | 250 | 3 | 5 | 5 | 5 | 5 |
| 1.014 | 250 | 5 | 5 | 5 | 5 | 5 |
| 1.015 | 250 | 5 | 5 | 5 | 5 | 2 |
| 1.016 | 250 | 5 | 5 | NT | 5 | 5 |
| 1.017 | 250 | 5 | 5 | NT | 5 | 5 |
| 1.018 | 250 | 5 | 5 | NT | 5 | 5 |
| 1.019 | 250 | 5 | 5 | NT | 5 | 5 |
| 1.020 | 250 | 5 | 5 | NT | 5 | 5 |
| 1.021 | 250 | 5 | 5 | NT | 5 | 5 |
| 1.022 | 250 | 5 | 5 | NT | 5 | 5 |
| 1.023 | 250 | 5 | 5 | NT | 5 | 5 |
| 1.024 | 250 | 5 | 5 | NT | 5 | 5 |
| 1.025 | 250 | 5 | 5 | NT | 5 | 5 |
| 1.026 | 250 | 5 | 5 | NT | 5 | 5 |
| 1.027 | 250 | 5 | 5 | NT | 5 | 5 |
| 1.028 | 250 | 5 | 5 | NT | 5 | 5 |
| 1.029 | 250 | 5 | 5 | NT | 4 | 5 |
| 1.030 | 250 | 3 | 3 | NT | 1 | 2 |
| 1.031 | 250 | 5 | 5 | NT | 5 | 5 |
| 1.032 | 250 | 5 | 5 | NT | 5 | 5 |
| 1.033 | 250 | 5 | 5 | NT | 5 | 5 |
| 1.034 | 250 | 5 | 5 | 5 | 4 | 3 |
| 1.035 | 250 | 5 | 5 | 5 | 5 | 5 |
| 1.036 | 250 | 1 | 1 | 1 | 1 | 1 |
| 1.037 | 250 | 5 | 5 | 5 | 5 | 5 |
| 1.038 | 250 | 5 | 5 | 5 | 5 | 5 |
| 1.039 | 250 | 5 | 5 | 5 | 5 | 5 |
| 1.040 | 250 | 1 | NT | 1 | 1 | 1 |
| 1.041 | 250 | 5 | 2 | 1 | 1 | 3 |
| 1.042 | 250 | 1 | 2 | NT | 1 | 1 |
| 1.043 | 250 | 5 | 5 | NT | 5 | 5 |
| 1.044 | 250 | 1 | 1 | NT | 1 | 1 |
| 1.045 | 250 | 1 | 1 | NT | 1 | 2 |
| 1.046 | 250 | 5 | 5 | NT | 5 | 5 |
| 1.047 | 250 | 5 | 5 | 5 | 5 | 5 |
| 1.048 | 250 | 5 | 5 | 4 | 4 | 5 |
| 1.019 | 250 | 5 | 5 | 5 | 4 | 5 |
| 1.050 | 250 | 5 | 5 | 5 | 5 | 5 |
| 1.051 | 250 | 4 | 5 | 5 | 5 | 5 |
| 1.052 | 250 | 5 | 4 | 5 | 4 | 3 |
| 1.053 | 250 | 5 | 5 | 5 | 5 | 5 |
| 1.054 | 250 | 5 | 5 | 5 | 5 | 5 |
| 1.055 | 250 | 5 | 5 | 5 | 5 | 5 |
| 1.056 | 250 | 3 | 3 | 3 | 3 | 1 |
| 1.057 | 250 | 1 | 1 | 1 | 1 | 1 |
| 1.058 | 250 | 5 | 4 | 5 | 5 | 2 |
| 1.059 | 250 | 5 | 5 | 5 | 5 | 4 |
| 1.060 | 250 | 5 | 5 | 5 | 5 | 4 |
| 1.061 | 250 | 3 | 5 | 1 | 1 | 2 |
| 1.062 | 250 | 1 | 1 | NT | 1 | 1 |
| 1.063 | 250 | 1 | 1 | NT | 1 | 1 |
| 1.064 | 250 | 5 | 2 | 5 | 3 | 1 |
| 1.065 | 250 | 5 | 5 | 5 | 4 | 4 |
| 1.066 | 250 | 5 | 5 | 5 | 5 | 5 |
| 1.067 | 250 | 5 | 5 | 5 | 5 | 5 |
| 1.068 | 250 | 5 | 5 | 5 | 4 | 5 |
| 1.069 | 250 | 5 | 5 | 5 | 5 | 5 |
| 1.070 | 250 | 5 | 5 | 5 | 5 | 5 |
| 1.071 | 250 | 5 | 5 | 5 | 5 | 5 |
| 1.072 | 250 | 5 | 5 | 5 | 5 | 5 |
| 1.073 | 250 | 5 | 5 | 5 | 5 | 5 |
| 1.074 | 250 | 1 | 1 | 1 | 1 | 1 |
| 1.075 | 250 | 4 | 1 | 5 | 2 | 1 |

NT = Not tested

1. A compound of Formula (I): 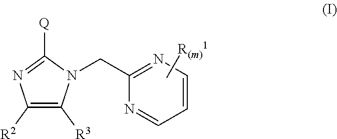
or an agronomically acceptable salt thereof,
wherein
Q is phenyl or a C-linked 6-membered heteroaryl wherein said phenyl or 6-membered heteroaryl is optionally substituted by one or more R⁴;
R¹ is independently selected from the group consisting of halogen, —CN, NO₂, C₁-C₄alkyl, C₁-C₄haloalkyl, C₃-C₆cycloalkyl, C₂-C₄alkenyl, C₂-C₄alkynyl, —S(O)ₚC₁-C₄alkyl, C₁-C₄alkoxy-, —C(O)C₁-C₄alkyl, —C(O)OC₁-C₄alkyl, C₁-C₄haloalkoxy and C₁-C₄alkoxyC₁-C₃alkyl-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —S(O)ₚC₁-C₄alkyl and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁵R⁶;
R⁴ is selected from the group consisting of halogen, C₁-C₄ alkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁵R⁶;
R⁵ is hydrogen or C₁-C₄alkyl;
R⁶ is hydrogen or C₁-C₄alkyl;
m=1 or 2; and
p=0, 1 or 2.

2. A compound of Formula (I) according to claim 1, which is of Formula (Ia) 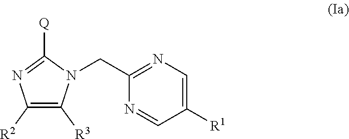
wherein Q, R¹, R² and R³ are as defined in claim 1 above.

3. A compound according to claim 1 wherein R³ is hydrogen.

4. A compound according to claim 1, wherein R¹ is chloro.

5. A compound according to claim 1, wherein R² is —CF₃ or —CF₂H.

6. A compound according to claim 1, wherein Q is selected from the group consisting of: 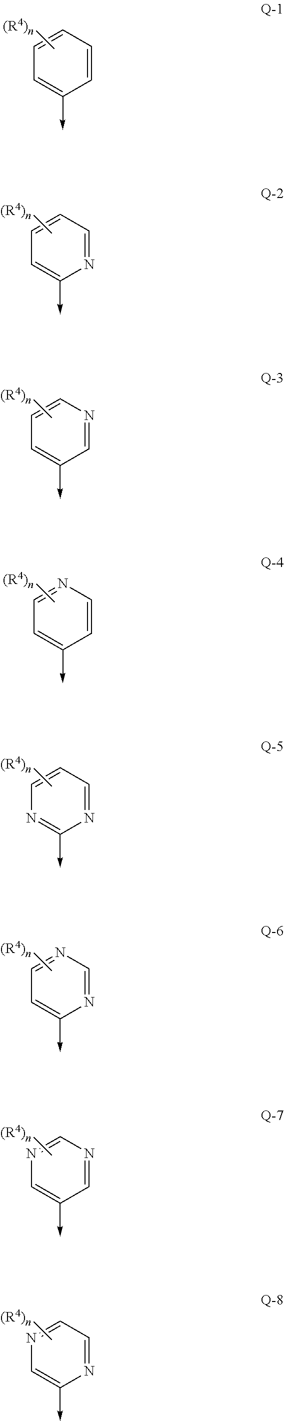 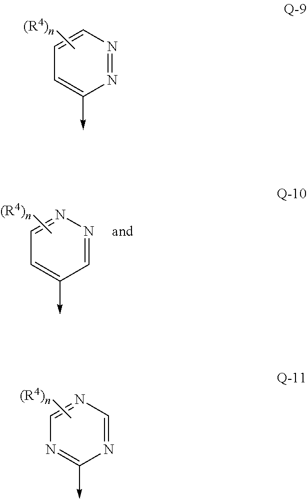
wherein n is 0, 1 or 2.

7. A compound according to claim 1, wherein Q is selected from the group consisting of Q-1, Q-3 and Q-7.

8. A compound according to claim 7, wherein n is 1.

9. A compound according to claim 8, wherein R⁴ is selected from the group consisting of cyano, methyl, halogen and —CF₃.

10. A compound according to claim 1, wherein Q is 4-Cl-phenyl-.

11. An herbicidal composition comprising a compound according to claim 1 and an agriculturally acceptable formulation adjuvant.

12. An herbicidal composition according to claim 11, further comprising at least one additional pesticide.

13. An herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as an herbicide.
